(19) United States
(12) Patent Application Publication
(10) Pub. No.: US 2024/0153608 A1
(43) Pub. Date: May 9, 2024

(54) Systems, Methods, Mediums, and Apparatuses for Capturing Medications and Medication Usage

(71) Applicant: Johnson & Johnson Consumer Inc., Skillman, NJ (US)

(72) Inventors: Liyuan Ma, North Wales, PA (US); Joshua Ide, Clarks Summit, PA (US)

(21) Appl. No.: 18/386,194

(22) Filed: Nov. 1, 2023

Related U.S. Application Data
(60) Provisional application No. 63/422,824, filed on Nov. 4, 2022.

(51) Int. Cl. G16H 20/10; G16H 70/40

(52) U.S. Cl. CPC G16H 20/10; G16H 70/40

(57) ABSTRACT

Systems, methods, non-transitory computer-readable mediums, and/or apparatuses are provided for determining a medication used by a patient. Label information for a medication may be determined. A source national drug code (NDC) associated with the medication may be determined. The source NDC may be validated. A product NDC and a normalized identifier may be determined. A brand name associated with the medication may be determined. One or more active ingredients associated with the brand name may be determined. A drug display name may be determined. The drug display name and active ingredient information for the one or more active ingredients may be displayed. A summary indicating an amount of the active ingredient may be displayed, for example, based on a medication diary.

### CROSS-REFERENCE TO RELATED APPLICATIONS

This application claims the benefit of U.S. Provisional Patent Application No. 63/422,824, filed Nov. 4, 2022, the disclosure of which is incorporated herein by reference in its entirety.

### BACKGROUND

Medicines play a role (e.g., a vital role) for the treatment and management of diseases and symptoms. A record (e.g., a complete and accurate record) of patient medications may be important for the safe and effective use of medicines. However, discrepancies may be common among both recorded and reported medications. Such discrepancies may lead to medication errors. Reducing medication discrepancies and errors may be important for achieving medication safety (e.g., further medication safety).

### SUMMARY

Systems, methods, non-transitory computer-readable mediums, and/or apparatuses may be provided for capturing medications and/or medication usage. For example, a device and/or method performed by the device may be provided for determining a medication used by a patient. Label information for the medication may be determined. A first message may be sent to a first server. The first message may indicate a request for a first medication identifier, such as a first national drug code (NDC) that may be associated with the medication. The first message may indicate the label information. The first NDC may be in a first format. A second message may be received from the first server. The second message may indicate the first NDC and may indicate a RxNorm concept unique identifier (RxCUI). A second NDC may be determined using the first NDC. The second NDC may be in a second format. The second format may be different from the first format. A brand name may be determined using the second NDC. One or more active ingredients may be determined using the RxCUI. The one or more active ingredients may be associated with the brand name. A drug display name may be determined using the brand name and the one or more active ingredients. Active ingredient information may be determined for the one or more active ingredients. The drug display name and the active ingredient information may be displayed to a user, such as the patient.

Systems, methods, non-transitory computer-readable mediums, and/or apparatuses may be provided for capturing medications and/or medication usage. For example, a device and/or method performed by the device may be provided for determining a medication and a medication usage associated with a patient. Label information for the medication may be determined. A first message may be sent to a first server. The first message may indicate a request for a first medication identifier, such as a first national drug code (NDC), that may be associated with the medication. The first message may indicate the label information. The first NDC may be in a first format. A second message may be received from the first server. The second message may indicate the first NDC and may indicate a RxNorm concept unique identifier (RxCUI). A second NDC may be determined using the first NDC. The second NDC may be in a second format. The second format may be different from the first format. A brand name may be determined using the second NDC. One or more active ingredients may be determined using the RxCUI. The one or more active ingredients may be associated with the brand name. A drug display name may be determined using the brand name and the one or more active ingredients. A usage associated with the medication and a user may be determined. The user may be the patient. Active ingredient information may be determined for the one or more active ingredients. The drug display name, the usage, and the active Ingredient information may be displayed to a user, such as the patient.

Systems, methods, non-transitory computer-readable mediums, and/or apparatuses may be provided for capturing medications and/or medication usage. Label information for a medication may be determined. A source national drug code (NDC) may be determined using the label information. The source NDC may be associated with the medication. A first message may be sent to a first server. The first message may indicate a request for a validation of the source NDC. A second message may be received from the first server. The second message may indicate the source NDC is valid, may indicate a product NDC, and may indicate a normalized identifier. A brand name associated with the medication may be determined using the product NDC. One or more active ingredients associated with the brand name may be determined using the normalized identifier. A drug display name may be determined using the brand name and the one or more active ingredients. Active ingredient information may be determined for the one or more active ingredients. The drug display name and the active ingredient information may be displayed to a user.

Systems, methods, non-transitory computer-readable mediums, and/or apparatuses may be provided for summarizing medication usage associated with a patient. A first normalized identifier may be determined using a first national drug code (NDC). The first normalized identifier may be associated with a first medication. A second normalized identifier may be determined using a second NDC. The second normalized identifier may be associated with a second medication. The first normalized identifier may be used to determine that the first medication comprises a first amount of an active ingredient. The second normalized identifier may be used to determine that second medication comprises a second amount of an active ingredient. A medication summary may be displayed. The medication summary may comprise a drug name associated with the active ingredient, and a sum of the first amount and the second amount of the active ingredient. In examples, the medication summary may be referred to as a dosage summary by active ingredient.

### BRIEF DESCRIPTION OF THE DRAWINGS

FIG. 1 depicts an example functional block diagram of the electrical components of an example smart device for capturing medications and/or medication usage.

FIG. 2A depicts an example architecture diagram for an example system to support a smart device.

FIG. 2B is a messaging flow diagram for the example system.

FIG. 3 depicts a block diagram of a system that may include one or more modules (e.g., software modules such as medication information, a medication list, and a dosage summary) for capturing medications and/or medication usage.

FIG. 4 depicts an example format for a National Drug Code (NDC).

FIG. 5 depicts an example method for capturing medications and/or medication usage.

FIG. 6 depicts another example method for capturing medications and/or medication usage.

FIG. 7 depicts an example of information regarding a medication that may be displayed to a user.

FIG. 8 depicts an example method for determining an amount of an active ingredient in a medication.

FIG. 9A depicts an example user interface that lists one or more identified medications.

FIG. 9B depicts an example user interface that summarizes one or more active ingredients associated with one or more medications.

FIG. 9C depicts an example user interface that summarizes one or more active ingredients associated with one or more medications.

FIG. 9D depicts an example user interface that provides a daily dosage summary for one or more active ingredients associated with one or more medications.

FIG. 10 depicts an example user interface that lists one or more medications associated with a patient.

FIG. 11 depicts example user interfaces for capturing medications and/or medication usage.

FIG. 12 depicts an example user interface for capturing medications and/or medication usage for a medication list.

FIG. 13 depicts an example user interface for capturing medications and/or medication usage for a drug interaction.

FIG. 14 depicts an example medication information.

FIG. 15 depicts an example user interface for capturing medications and/or medication usage for a medication diary.

FIG. 16 depicts an example user interface for capturing medications and/or medication usage for a medication diary.

FIG. 17 depicts an example user interface for capturing medications and/or medication usage for a dose assessment or an exposure assessment.

FIG. 18 depicts an example user interface for capturing medications and/or medication usage for a medication watch list.

FIG. 19 depicts an example user interface for capturing medications and/or medication usage for a drug interaction.

FIG. 20 depicts an example user interface for capturing medications and/or medication usage for a medication watch list.

### DETAILED DESCRIPTION

Features described herein may include developing a patient-centric mobile application to improve the capture of medications and usage.

Systems, methods, non-transitory computer-readable mediums, and/or apparatuses may be provided for capturing medications and/or medication usage. For example, a device and/or method performed by the device may be provided for determining a medication used by a patient. Label information for the medication may be determined. A first message may be sent to a first server. The first message may indicate a request for a first medication identifier, such as a first national drug code (NDC) associated with the medication. The first message may indicate the label information. The first NDC may be in a first format. A second message may be received from the first server. The second message may indicate the first NDC and may indicate a RxNorm concept unique identifier (RxCUI). A second NDC may be determined using the first NDC. The second NDC may be in a second format. The second format may be different from the first format. A brand name may be determined using the second NDC. One or more active ingredients may be determined using the RxCUI. The one or more active ingredients may be associated with the brand name. A drug display name may be determined using the brand name and the one or more active ingredients. Active ingredient information may be determined for the one or more active ingredients. The drug display name and the active ingredient information may be displayed to a user, such as the patient.

Systems, methods, non-transitory computer-readable mediums, and/or apparatuses may be provided for capturing medications and/or medication usage. For example, a device and/or method performed by the device may be provided for determining a medication and medication usage associated with a patient. Label information for the medication may be determined. A first message may be sent to a first server. The first message may indicate a request for a first medication identifier, such as a first national drug code (NDC), that may be associated with the medication. The first message may indicate the label information. The first NDC may be in a first format. A second message may be received from the first server. The second message may indicate the first NDC and may indicate a RxNorm concept unique identifier (RxCUI). A second NDC may be determined using the first NDC. The second NDC may be in a second format. The second format may be different from the first format. A brand name may be determined using the second NDC. One or more active ingredients may be determined using the RxCUI. The one or more active ingredients may be associated with the brand name. A drug display name may be determined using the brand name and the one or more active ingredients. A usage associated with the medication and a user may be determined. The user may be the patient. Active ingredient information may be determined for the one or more active ingredients. The drug display name, the usage, and the active ingredient information may be displayed to a user, such as the patient.

Systems, methods, non-transitory computer-readable mediums, and/or apparatuses may be provided for capturing medications and/or medication usage. Label information for a medication may be determined. A source national drug code (NDC) may be determined using the label information. The source NDC may be associated with the medication. A first message may be sent to a first server. The first message may indicate a request for a validation of the source NDC. A second message may be received from the first server. The second message may indicate the source NDC may be valid, may indicate a product NDC, and may indicate a normalized identifier. A brand name associated with the medication may be determined using the product NDC. One or more active ingredients associated with the brand name may be determined using the normalized identifier. A drug display name may be determined using the brand name and the one or more active ingredients. Active ingredient information may be determined for the one or more active ingredients. The drug display name and the active ingredient information may be displayed to a user.

In an example, the normalized identifier may be a RxNorm concept unique identifier (RxCUI).

In an example, the product NDC may comprise a labeler code and a product code.

In an example, the source NDC may be a package NDC, and the package NDC may comprise a labeler code, a product code, and a package code.

In an example, the source NDC may be in a 10-digit format or an 11-digit format. The product NDC may be in a 9-digit format.

In an example, the label information for the medication may be determined by receiving data from a user. The data may include at least one of the source NDC, an active ingredient, a brand name, an image, or a video.

In an example, the label information for the medication may be determined by determining an image or a video of a medication label. At least one of the source NDC, an active ingredient, or a brand name may be determined from the image using optical character recognition.

In an example, the brand name may be determined using the product NDC by sending a brand name request message to a second server. The brand name request message may indicate a request for a brand name associated with the product NDC. A brand name response message may be received from the second server. The brand name response message may indicate the brand name.

In an example, one or more active ingredients associated with the brand name may be determined by sending an ingredient request message to the first server. The ingredient request message may indicate a request for one or more active ingredients associated with the normalized identifier. An ingredient response message may be received from the first server. The ingredient response message may indicate the normalized identifier and one or more active ingredients. The one or more active ingredients may be within a database such that the one or more active ingredients are associated with the brand name.

In an example, the drug display name may be determined using the brand name and the one or more active ingredients by determining a prescribable synonym name for at least an active ingredient of the one or more active ingredients. The drug display name may be determined by combining the brand name with the prescribable synonym name.

In an example, the active ingredient information for the one or more active ingredients may be determined by sending an active ingredient information request to a third server. The active ingredient information request may indicate at least one of a structured product labeling identification (SPL ID) or the normalized identifier. An active ingredient information response may be received. The active ingredient information response may indicate an active ingredient. The active ingredient information response may indicate at least a warning, a direction regarding the active ingredient, a usage direction, or a drug interaction warning.

In an example, the active ingredient information may comprise at least an active ingredient from one or more active ingredients, a dose and/or strength associated with the active ingredient, a dose unit associated with the active ingredient, or a unit type associated with the medication. As used herein, the term dose may be used interchangeably with the term strength.

Systems, methods, non-transitory computer-readable mediums, and/or apparatuses may be provided for summarizing medication usage associated with a patient. A first normalized identifier may be determined using a first national drug code (NDC). The first normalized identifier may be associated with a first medication. A second normalized identifier may be determined using a second NDC. The second normalized identifier may be associated with a second medication. The first normalized identifier may be used to determine that the first medication comprises a first amount of an active ingredient. The second normalized identifier may be used to determine that the second medication comprises a second amount of an active ingredient. A medication summary may be displayed. The medication summary may comprise a drug name associated with the active ingredient and a sum of the first and second amounts of the active ingredient.

In an example, a minimum amount of the active ingredient consumed by the patient may be determined, using the first and second amounts.

In an example, the maximum amount of the active ingredient consumed by the patient may be determined using the first and second amounts.

In an example, an average amount of the active ingredient consumed by the patient may be determined, using the first and second amounts.

In an example, the drug name may be a first drug name, and the active ingredient may be a first active ingredient. The first medication may comprise a third amount of a second active ingredient, which may be identified using the first normalized identifier.

In an example, a first usage of the first medication may be determined. The first usage may indicate when the patient consumed the first medication.

In an example, the medication summary may further comprise the first usage and a second sum. The second sum may be the third amount of the second active ingredient.

In an example, the medication summary may further comprise a first medication information associated with the first active ingredient and a second medication information associated with the second active ingredient.

In an example, the drug name may be a first drug name, the active ingredient may be a first active ingredient, and the sum may be a first sum. The first normalized identifier may be used to determine that the first medication comprises a third amount of a second active ingredient. The second normalized identifier may be used to determine that the second medication comprises a fourth amount. The second normalized identifier may be used to determine that the second medication comprises a fifth amount of a third active ingredient.

In an example, a first usage of the first medication may be determined. The first usage may indicate when the patient consumed the first medication. A second usage of the second medication may be determined. The second usage may indicate when the patient consumed the second medication.

In an example, the sum may be a first sum, and the medication summary may further comprise the first usage, the second usage, a second sum, and a third sum. The second sum may be the third amount of the second active ingredient and the fourth amount of the second active ingredient. The third sum may be the fifth amount of the third active ingredient.

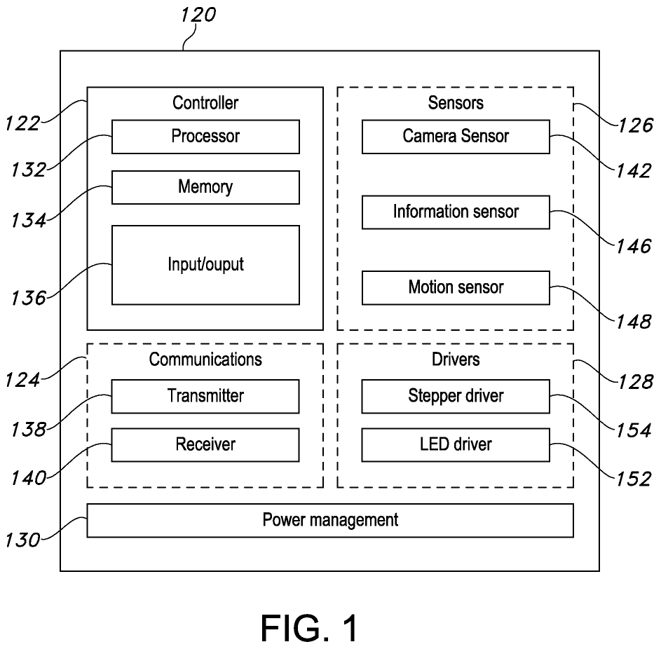

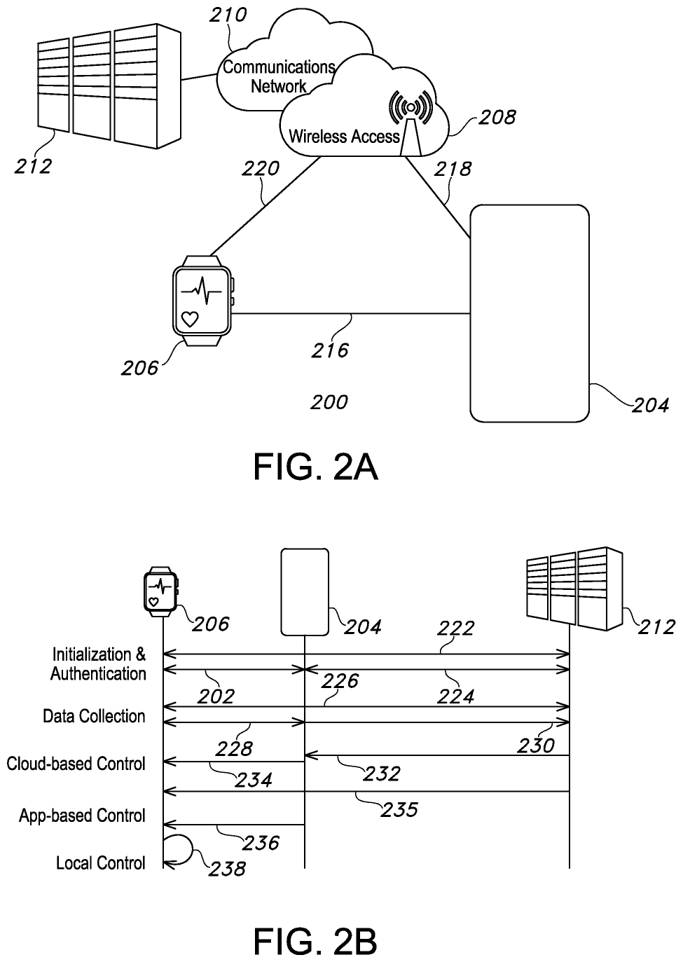

FIG. 1 depicts an example functional block diagram of electrical components of an example smart device for capturing medications and/or medication usage. For example, FIG. 1 may depict an example functional block diagram of electrical components of an example smart device for capturing medications associated with a patient as well as how a patient may use those medications. The smart device may be a smartphone (e.g., iOS®, Android®, and the like), a tablet (e.g., an iPad, a Galaxy Tablet®, and the like), a smartwatch (e.g., an Apple Watch®, a Google Pixel Watch, and the like), a wearable device, a cellular phone, a computer, a server, and/or the like. Components 120 may be incorporated into the smart device, such as devices 204 and 206 (shown with respect to FIG. 2), and/or may be incorporated into a computing resource, such as 212 (also shown with respect to FIG. 2). Referring again to FIG. 1, the components 120 may integrate sensing, electromechanical driving, communications, and/or digital-processing functionality to the structure and operation of the device. In examples, the components 120 may include a controller 122, communications interfaces 124, sensors 126, electrical and electromechanical drivers 128, and a power management subsystem 130.

Controller 122 may include a processor 132, a memory 134, and one or more input/output devices 136, for example. Controller 122 may be any suitable microcontroller, microprocessor, field programmable gate array (FPGA), application-specific integrated circuit (ASIC), or the like that is suitable for receiving data, computing, storing, and driving output data and/or signals. Controller 122 may be a device suitable for an embedded application. For example, controller 122 may include a system on a chip (SOC).

Processor 132 may include one or more processing units. Processor 132 may be a processor of any suitable depth to perform the digital processing requirements disclosed herein. For example, processor 132 may include a 4-bit processor, a 16-bit processor, a 32-bit processor, a 64-bit processor, or the like. The processor may be or may include a graphic processing unit (GPU), an artificial intelligence (AI) processing unit, a machine learning processing unit, and/or another processor that may be appropriate for graphic processing, AI, and/or machine learning.

Memory 134 may include any component or collection of components suitable for storing data. For example, memory 134 may include volatile and/or nonvolatile memory. Memory 134 may include random-access memory (RAM), read-only memory (ROM), erasable programmable read-only memory (EPROM), (electrically erasable programmable read-only memory) EEPROM, flash memory, or the like.

Input/output devices 136 may include any devices suitable for receiving and/or sending information. This information may be in the form of digitally encoded data (from other digital components, for example) and/or analog data (from analog sensors, for example). Input/output devices 136 may include serial input/output ports, parallel input/output ports, universal asynchronous receiver transmitters (UARTs), discrete logic input/output pins, analog-to-digital converters, and digital-to-analog converters. Input/output devices 136 may include specific interfaces with computing peripherals and support circuitry, such as timers, event counters, pulse width modulation (P generators, watchdog circuits, clock generators, and the like. Input/output devices 136 may provide communication within and among the components 120, for example, communication between the controller 122 and sensors 126, between the controller 122 and drivers 128, between the controller 122 and communications interfaces 124, and between controller 122 and power management subsystem 130, and as a conduit for any other combination of components 120. Components 120 may also support direct communication, for example, between sensor 126 and power management subsystem 130.

Communications interfaces 124 may include transmitter 138 and/or receiver 140. Communication interfaces 124 may include one or more transmitters 138 and/or receivers 140. Transmitter 138 and receiver 140 may include any electrical components suitable for communication to and/or from the electrical components 120. For example, transmitter 138 and receiver 140 may provide wireline communication and/or wireless communication to devices external to components 120 and/or external to the device within which components 120 are integrated.

Transmitter 138 and receiver 140 may enable wireline communication using any suitable communications protocol, for example, protocols suitable for embedded applications. For example, transmitter 138 and receiver 140 may be configured to enable universal serial bus (USB) communication, Ethernet local-area networking (LAN) communications, and the like.

Transmitter 138 and receiver 140 may enable wireless communications using any suitable communications protocol, for example, protocols suitable for embedded applications. For example, transmitter 138 and receiver 140 may be configured to enable a wireless personal area network (PAN) communications protocol, a wireless LAN communications protocol, a wide area network (WAN) communications protocol, and the like. Transmitter 138 and receiver 140 may be configured to communicate via Bluetooth, for example, with any supported or custom Bluetooth version and/or with any supported or custom protocol, including, for example, A/V Control Transport Protocol (AVCTP), A/V Distribution Transport (AVCTP), Bluetooth Network Encapsulation Protocol (BNEP), IrDA Interoperability (IrDA), Multi-Channel Adaptation Protocol (MCAP), and RF Communications Protocol (RFCOMM), and the like. In examples, transmitter 138 and receiver 140 may be configured to communicate via Bluetooth Low Energy (LE) and/or a Bluetooth Internet of Things (IoT) protocol. Transmitter 138 and receiver 140 may be configured to communicate via local mesh network protocols such as ZigBee, Z-Wave, Thread, and the like. Such protocols may enable the transmitter 138 and receiver 140 to communicate with nearby devices such as the user's cell phone and/or a smartwatch. Communication with a local networked device, such as a mobile phone, may enable further communication with other devices across a wide area network (WAN) to remote devices, on the Internet, on a corporate network, and the like.

Transmitter 138 and receiver 140 may be configured to communicate via LAN protocols, such as 802.11 wireless protocols, like Wi-Fi, including but not limited to communications in the 2.4 GHz, 5 GHz, and 60 GHz frequency bands. Such protocols may enable transmitter 138 and receiver 140 to communicate with local network access points, such as a wireless router in a user's home or office. Communication with a local network access point may enable further communication with other devices present on the local network or across a WAN to remote devices, on the Internet, on a corporate network, and the like.

The transmitter 138 and receiver 140 may be configured to communicate via mobile wireless protocols such as global system for mobile communications (GSM), 4G long-term evolution protocol (LTE), 5G, and 5G new radio (NR), and any variety of mobile Internet of things (IoT) protocols. Such protocols may enable the transmitter 138 and receiver 140 to communicate more readily, for example, when a user is mobile, traveling away from home or office, and without manual configuration.

The sensors 126 may include any device suitable for sensing an aspect of its environment, such as physical, chemical, mechanical, electrical, encoded information, and the like. Controller 122 may interact with one or more sensors 126. The sensors 126 may include, for example, camera sensor 142, information sensor 146, motion sensor 148, and the like. Although not shown, sensors 126 may include one or more biometric sensors, such as a heart rate sensor, a blood oxygen sensor, a blood pressure sensor, a combination thereof, and/or the like.

Camera sensor 142 may include any sensor suitable for capturing and/or recording an image and/or video. Camera sensor 142 may be a charged-couple device (CCD), an active-pixel sensor, a complementary metal-oxide-semiconductor (CMOS) sensor, an N-type metal-oxide-semiconductor (NMOS) sensor, or the like. In examples, camera sensor 142 may be used to take an image and/or video of information that may be encoded on a medication container (e.g., a medication label). The information may be encoded on the medication contain using text, characters, numbers, a quick read (QR) code, a readable integrated circuit (e.g., a one-wire identification chip), a near-field communications (NFC) tag, in physical/mechanical keying, in a Subscriber Identification Module (SIM), or the like. For example, a user may use camera sensor 142 to take an image of a medication label, and the text of the medication label may be processed by device 120.

The information sensor 146 may include any sensor suitable for reading stored information. In an embedded application with a physical platform, information may be encoded and stored on a variety of media that may be incorporated into aspects of physical design. For example, information about the authenticity, concentration, volume, etc. of a medication that may be consumed by a user and/or may be associated with the device. In examples, the information may be encoded on a medication container (e.g., a medication label) using text, characters, numbers, a quick read (QR) code, a data matrix, a readable integrated circuit (e.g., a one-wire identification chip), a near-field communications (NFC) tag, radio frequency identification (RFID), in physical/mechanical keying, in a Subscriber Identification Module (SIM), or the like. The user may use the device to scan a QR code, and the device may communicate the information to controller 122 via communications interface 124. In examples, information sensor 146 may also be suitable for writing information back onto a medium associated with the readable code, such as with a read/writable NFC tag, for example.

Motion sensor 148 may include any sensor suitable for determining relative motion, acceleration, velocity, orientation, and/or the like of the device. Motion sensor 148 may include a piezoelectric, piezoresistive, and/or capacitive component to convert physical motion into an electrical signal. For example, motion sensor 148 may include an accelerometer. Motion sensor 148 may include a microelectromechanical system (MEMS) device, such as a MEMS thermal accelerometer. Motion sensor 148 may be suitable for sensing a repetitive or periodic motion, such as fidgeting by a user holding or wearing the device. Motion sensor 148 may communicate this information via input/output devices 136 to processor 132 for processing.

The device may include one or more drivers 128 to communicate feedback to a user and/or to drive a mechanical action. Drivers 128 may include a light-emitting diode (LED) driver 152, stepper driver 154, and the like. Other drivers 128 may include haptic feedback drivers, audio output drivers, heating element drivers, and/or the like.

LED driver 152 may include any circuitry suitable for illuminating an LED. LED driver 152 may be controllable by the processor 132 via the input/output devices 136. LED driver 152 may be used to indicate status information to a user. LED driver 152 may include a multicolor LED driver.

Stepper driver 154 may include any circuitry suitable for controlling a stepper motor. Stepper driver 154 may be controllable by the processor 132 via the input/output driver 136. Stepper driver 154 may be used to control a stepper motor associated with a medical device. In an example, the stepper driver 154 may be used to control a stepper motor of an insulin pump. In an example, the stepper driver 154 may be used to control a motor of a prosthetic arm.

Power management subsystem 130 may include circuitry suitable for managing and distributing power to the components of smart device 120. Power management subsystem 130 may include a battery, a battery charger, a direct current (DC) power distribution system, and the like. Power management subsystem 130 may communicate with the processor 132 via input/output devices 136 to provide information such as battery charging status. Power management subsystem 130 may include a replaceable battery and/or a physical connector to enable external charging of the battery.

FIG. 2A depicts an example architecture diagram for an example system to support a device, such as a smart device. System 200 may include a smartphone 204 with a corresponding application (e.g., an app), a smartwatch 206 with a corresponding app, a wireless access network 208, a communications network 210, and a computing resource 212.

The smart device may be a smartphone, a tablet (e.g., an iPad), a smartwatch, a wearable device, a cellular phone, a computer, a server, and/or the like. The smart device may be smart device shown with respect to FIG. 1 and may be smartwatch 206 (e.g., an Apple Watch®, a Google Pixel Watch®, and the like), smartphone 204 (e.g., iPhone®, Samsung Galaxy 523®, and the like), and/or computing resource 212 shown with respect to FIGS. 2A-B.

Referring again to FIG. 2A, smartphone 204 may include an app for capturing medications and/or medication usage. Smartphone 204 may provide passive or active tracking and/or location services. Smartphone 204 may collect data regarding the user, process data regarding the user, and/or share data regarding the user. For example, smartphone 204 may be able to use one of its sensors to collect information regarding medication and may be able to share that data with smartwatch 206 and/or computing resource 212. As another example, smartphone 204 may be capable of determining that a user has used medication and may be able to share that data with smartwatch 206 and/or computing resource 212.

Smartwatch 206 may be able to provide a list of medications to a user. The list of medications may be a list of medications the user may use and/or may have used. The list of medications may be a list of active ingredients found in the medications used by the user. Smartwatch 206 may provide biometric feedback and data such as heart rate and/or heart rate variability. Smartwatch 206 may perform activity tracking and may provide activity information. Smartwatch 206 may be used by a user to add a medication to a list of medications and remove a medication from the list.

The computing resource 212 may provide data storage and processing functionality. The computing resource 212 may receive and analyze medication data and/or medication usage data. For example, computing resource 212 may receive and analyze a medication used by a user and may determine what active ingredients are contained in the medication and what amount of those active ingredients was consumed.

The components of system 200 may communicate with each other over various communications protocols. Smartwatch 206 may communicate with the smartphone 204 over a link, such as Bluetooth wireless link 216. Smartphone 204 may communicate with the wireless access network 208 over a link, such as wireless link 218. Smartwatch 206 may communicate with the wireless access network 208 over a link, such as wireless link 220. Wireless link 218 and/or wireless link 220 may include any suitable wireless protocol, such as 802.11 wireless protocols like Wi-Fi, GSM, 4G LTE, 5G, and 5G NR, any mobile IoT protocols, or the like.

Communication network 210 may include a long-distance data network, such as a private corporate network, a virtual private network (VPN), a public, commercial network, an interconnection of networks, such as the Internet, or the like. The communication network 210 may provide connectivity to computing resource 212.

Computing resource 212 may include any server resources suitable for remote processing and/or storing of information. For example, computing resource 212 may include a server, a cloud server, a data center, a virtual machine server, and/or the like. In examples, the smartwatch 206 may communicate with the computing resource 212 via its own wireless link 220, and the smartwatch 206 may communicate with the computing resource 212 via its own wireless link 218.

System 200 may enable the collection and processing of information related to a medication associated with a user (e.g., a patient). For example, system 200 may capture the medication a user may be using, when a medication is being used by a user, how much of a medication a user may be consuming, a combination thereof, and/or the like. System 200 may allow a user to track what medication they may be taking. System 200 may determine how much of an active ingredient, which may be a component of a medication, a user may be consuming.

For example, a user may consume a medication comprised of three active ingredients. The system 200 may determine that the user has consumed the medication and that the user has consumed 10 mg of the first active ingredient, 5 mg of a second active ingredient, and 4 mg of a third active ingredient.

As another example, the user may consume a first medication and a second medication. The first medication may be comprised of three active ingredients; 10 mg of a first active ingredient, 5 mg of a second active ingredient, and 4 mg of a third active ingredient. The second medication may be comprised of two active ingredients; 2 mg of the first active ingredient and 4 mg of the second active ingredient. System 200 may provide a summary of the active ingredients consumed by the user (e.g., the patient). For example, system 200 may calculate that the user has consumed 12 mg of the first active ingredient, 9 mg of the second active ingredient, and 4 mg of a third active ingredient.

FIG. 2B is an example messaging flow diagram for the example system 200. system 200 may include communication and processing for functions such as initialization and authentication of the smartphone 204 and/or medication app; data collection from a smartwatch 206 and/or smartphone 204; cloud base control, triggering, notification messaging, and the like, app-based control, messaging and notifications, and the like.

Initialization and authentication messaging 222 may be exchanged between one or more of smartwatch 206, smartphone 204, or computing resource 212. For example, a user may create a user account via the smartphone 204. The account information may be processed by computing resource 212. The new user may initialize smartwatch 206 to authenticate the smartwatch 206. The information may be communicated via messaging 202 to the smartphone 204 and then via initialization and authentication messaging 224 to computing resource 212. The information may be communicated via initialization and authentication messaging 222 to computing resource 212. Responsive information about user accounts, medication usage, medication consumption, medications associated with a user, and the like may be messaged back to the smartwatch 206 and/or smartphone 204.

Data collection functionality may be provided and may include messaging 226 from the smartwatch 206, smartphone 204, and/or computing resource 212. This messaging may include information such as activity information, heart rate, heart rate variability, medication consumption, medication information, electronic medication records, medical data regarding a patient, prescriptions, and/or the like. The data collection functionality may include messaging 228 from smartwatch 206 to smartphone 204. In examples, the smartphone 204 may aggregate messaging 228, process it locally, and/or communicate it or related information to the computing resource 212 via messaging 230.

System 200 enables cloud-based, app-based, and local-based control functions. For example, personalized medication data, medication data, medication consumption data, active ingredient data, statuses, and/or reporting may be provided from computing resource 212 to smartphone 204 via messaging 232, and if appropriate, from smartphone 204 to smartwatch 206 via messaging 234. Computing resource 212 may communicate directly to smartwatch 206 by using messaging 235.

For example, personalized medication data, statuses, and/or reporting may be generated from an application and displayed at smartphone 204 and smartwatch 206. The application may be on computing resource 212, smartphone 206, and/or smartwatch 206. The personalized medication data, statuses, and/or reporting may be communicated to smartwatch 206 via messaging 236.

In examples, the smartwatch 206 may provide local control via its local processor. Internal system calls and/or local messaging is illustrated as a local loop 238. For example, smartwatch 206 may provide personalized medication data, statuses, and/or reporting.

The features and examples described herein may include developing a patient-centric mobile application to improve the capture of medications and usage.

Medicines may play a role in the treatment and management of diseases and symptoms, and the safe use of medications requires, among other things, knowledge and consideration of one or more of the medications that the patient is taking or receiving to avoid omissions, duplications, dosing errors, and potential adverse interactions with new drugs being prescribed. However, discrepancies may be common among both recorded and reported medications. Discrepancies may occur regardless of the class of medication or the care setting. Such discrepancies may lead to medication errors. Reducing medication discrepancies and errors may improve medication safety.

Discrepancies between both recorded and reported medications are common and may involve all classes of medications and care settings. The discrepancies may occur across geographies globally. In example, patients in a primary care clinic in the United States may experience discrepancies. In an example, patients in a primary care center in Sweden may experience discrepancies. The reported medication discrepancies may occur in patients in a practice of cardiologists. Discrepancies may occur where patients do not report taking medications. In examples, 30% and 70% of patients may experience an error or an unintentional change to their medicines when they move from one care setting to another. Common reasons for these discrepancies may include the omission of medications (e.g., over the counter [OTC]), unfilled prescriptions, incorrect dosages, and/or the like. Medication discrepancy may lead to medication errors and adverse outcomes, may contribute to global health expenditures.

Medication reconciliation has been recognized by several international patient safety organizations, such as The Joint Commission (TJC), Institute for Healthcare Improvement (IHI), and the World Health Organization (WHO) for potential to achieve better medication safety and optimize transitions of care. In an example, a medication history may involve capturing and/or determining medications the patient is taking, which may include prescription, OTC, and complementary. In an example, a medication list may be created and/or determined where the medication list may include prescribed, OTC, and complementary medicines. In an example, medication reconciliation may be implemented in such a way as to reduce the time and resources requested of a patient to ensure that a patient may have an ability and willingness to provide the medication information accurately.

Examples provided herein may provide software for identifying and/or capturing medications (e.g., a mobile application) to help patients capture their medications, track usage, and track adherence, where some of the medications may be associated with a disease or product. The software may avoid requesting tedious manual entry of medications. The software may include features beyond pill reminders and adherence tracking. The software may assist in the standardization of medication capture given the large number of medications approved in the United States (e.g., >20,000 Rx, >300,000 OTC) with various active ingredients, strengths, and formulations. The software may help patients track their personal use of medications. The software may capture accurate drug information, such as ingredients, strength and formulation. The software may allow medication information to be shared with the healthcare providers, or to be aggregated for research purposes.

Ascertainment of medication exposure may be an element (e.g., a key element) in the generation of real-world evidence (e.g., for regulatory and clinical decision-making). Some healthcare systems may allow for (e.g., only allow for) the capture of prescription (Rx) medicines that a healthcare provider may have entered. But many existing healthcare systems may not allow for the capture of over the counter (OTC) medicines. In examples, healthcare systems often capture prescription medicines as medication orders or dispensing records, which may serve as proxies (e.g., medication exposure/patient adherence).

A self-care model and a patient-centric approach to healthcare may be provided. Self-care may be the ability of an individual, family, and/or community to promote health, to prevent disease, to maintain health, and/or to cope with an illness or disability with or without the support of a healthcare provider. Guidelines regarding self-care interventions for health may include recommendations on safe and effective interventions that may be used within an enabling environment (e.g., a supportive enabling environment).

Many patients who may be mobile and self-medicate may play a role (e.g., a central role) in creating and maintaining a medication list of the patients (e.g., as the patients may know the medications the patients are taking every day).

Self-care interventions may assist (e.g., empower) individuals and communities to manage the health and well-being of the individuals, strengthen national institutions (e.g., with efficient use of domestic resources for health), improve primary health care, and contribute to achieving universal health coverage. Assisting (e.g., empowering) individuals in managing their respective health may be a priority (e.g., important) as self-care (e.g., better self-care) may be an aspect of preventing a global epidemic (e.g., of chronic noncommunicable diseases). Self-care interventions may bring users greater choice, access, control, satisfaction, and affordable options to manage their respective health care needs. Self-care may recognize the strengths of individuals as active agents in the health care of the users and not passive recipients (e.g., not merely passive recipients) of health services.

The healthcare market may change (e.g., is changing) to adjust (e.g., keep pace) for modern needs (e.g., including self-treatment). Self-treatment may include patients diagnosing a condition and picking an appropriate medication from a pharmacy to treat the symptoms of the respective patients. Self-treatment may include allowing consumers to be actively engaged in their health. When patients self-treat, access to OTC medication may be relevant (e.g., of prime importance). Medications (e.g., many medications) available as OTC today may be previously labeled as prescription medications. Safety studies and trials may be conducted for drugs deemed appropriate for use without a prescription.

Person/patient-generated health data (PGHD) may provide data on exposures, outcomes, and covariates. They may provide an indication of a background risk of a patient (e.g., for an event of interest), more relevant endpoints (e.g., steps walked, hours slept), and measurements of real-time exposure (e.g., a sensor on an inhaler recording the dose and/or strength, geographic location, and the time and date of use).

As disclosed herein, embodiments may improve and/or simplify medication reconciliation for safer use of medicines. Healthcare interventions may be intended to benefit patients but may also cause harm. A combination (e.g., a complex combination) of processes, technologies, and human interactions that may include the modern healthcare delivery system may be beneficial. This combination of techniques, technologies, and human interactions may involve an inevitable risk of patient harm that may result in actual harm. Medication safety may (e.g., should) start with appropriate prescribing and a risk-benefit analysis (e.g., a thorough risk-benefit analysis) of a medication (e.g., each medication).

Identifying medication may be provided. Creating and maintaining a medication list for a patient may be provided. Creating and maintaining a medication list for every patient may represent a fulfillment of a responsibility (e.g., of physicians). Developing (e.g., properly developing) and maintaining a medication list (e.g., a correct medication list) for one or more patients may include delegation (e.g., appropriate delegation), implementation of relevant information technology, creation of systems to support work to maintain a list, and development of payment mechanisms and performance measures.

Medication reconciliation may be a process in which healthcare professionals may partner with patients to ensure medication information may be accurate.

The medication reconciliation process may assist (e.g., may be important) in facilitating medication and patient safety at transitions of care. Medication reconciliation may improve medication safety by identifying unintended medication discrepancies, especially at care transitions.

Features described herein may include extensibility and/or integration with Electronic Health Records (EHRs). EHRs (e.g., many EHRs) may be single-site EHRs or inpatient records (e.g., confined to inpatient records only). To improve medication safety at transitions, EHRs or summary care records may reflect a patient's medication usage across transitions, may be accessible (e.g., to patients and to treating health care professionals), and may be editable by one or more parties to improve completeness and accuracy.

Morbidities (e.g., multiple morbidities) may (e.g., usually may) be treated with multiple medications (polypharmacy), which may put a patient at risk of adverse drug events and drug interactions when not used appropriately. Multiple medicines or polypharmacy (e.g., the use of multiple medicines or polypharmacy) may be clinically appropriate. For example, patients with inappropriate polypharmacy may be at an increased risk of adverse events, and poor health outcomes may be identified.

Capturing (e.g., a robust capturing) of Rx/OTC Exposure for real-world data (RWD) and real-world evidence (RWE) may be provided. RWD (accurate world data) may be data that may relate to patient health status and/or the delivery of health care that may be collected from various sources. RWE may be evidence, such as clinical evidence, about a medical product's usage, potential benefits, and/or risks. RWE may be derived from an analysis of RWD. Examples of RWD may include data derived from EHRs, medical claims data, data from product and disease registries, patient-generated data from in-home use, and/or data gathered from other sources that may inform health status.

The potential utilization of RWE in health care decisions for prescribed medicines may have gained significant attention among industry, regulators, and professional societies. Comparatively, little focus has been on the potential role of RWE for non-prescription medicines. A number of differences may exist between prescription and non-prescription medicines in terms of the sources of RWD and a request for evidence, such as RWE. The potential utilization of patient-generated health data may be relevant for non-prescription medicines, given that consumers may assume the primary role in managing their condition without health care professional supervision and may also be at the center of the generation and distribution of patient-generated health data.

Improving adverse event reporting may be provided. For example, improving adverse event reporting with NDC Codes, pictures, and diary information may be provided. A medication identifier, such as NDC codes, may be helpful, as NDC codes may pertain to pharmacovigilance and drug safety. In an example, embodiments disclosed herein may be used to determine and/or report adverse events for a brand name, a prescription drug, a generic drug, an active ingredient, and/or the like.

A medical identifier (which may be referred to as a regulatory identifier) may be a code that a regulatory agency may provide so that the medical identifier may identify a medication. The medical identifier may be a national drug code. For example, the national drug code database may identify a medication using a medical identifier, which may be a National Drug Code (NDC). An NDC may allow products, such as medications, to be tracked (e.g., at a granular level). For example, an NDC may allow a medication to be tracked based on an active ingredient, a strength, packaging, a manufacturer, a combination thereof, and/or the like. An adverse event may be related to an active ingredient with a particular strength (e.g., Vioxx 50 mg) isolated to individual manufacturers due to excipients, quality control, or contamination.

In an example, features described herein may extend outside of the United States. Products may be identified differently in different regions. For example, medication identifiers, such as NDCs and/or RxCUIs, may vary according to country. For example, medication identifiers may be referred to as regulatory identifiers, as the identifiers may be provided by an authority regulating medications in a country. Table 1 describes identifier fields for supported regions, which may be used as medication identifiers.

TABLE 1

| | Region | | Local Product | |
|---|---|---|---|---|
| Region | Code | Product ID Field | ID | Example |
| Austria | at | at_product_id | MA number | 16965 |
| Canada | ca | dpd | DIN | 2341093 |
| Colombia | CO | co_product_id | Invima ID | INVIMA M-012422 |
| European | eu | ema_ma_number | EU MA | EU/1/04/276/001 |
| Union | | | number | |
| European | eu | ema_product_code | Product | EMEA/H/C/000471 |
| Union | | | number | |
| Indonesia | idn | idn_product_id | Nomor | GKL0122234837A1 |
| | | | registrasi | |
| Italy | it | it_product_id | AIC | 38835056 |
| Malaysia | my | my_product_id | Registration | MAL12715054ACZ |
| | | | no. | |
| Singapore | sg | sg_product_id | License no. | SIN11734P |
| Thailand | th | th_product_id | Registration | 1C 95/60 (N) |
| | | | no. | |
| Turkey | tr | tr_product_id | Barcode | 8699508150243 |
| United States | us | ndc | 9-digit NDC | 0169-5174 |
| United States | us | ndc | 10-digit NDC | 0169-5174-1 |
| United States | us | ndc | 11-digit NDC | 0169-5174-01 |

As disclosed herein, a medical identifier may be an identifier provided by a regulatory authority to identify a medication, a prescription medication, an over-the-counter medication, an active ingredient, a combination thereof, and/or the like.

Medication errors in a hospital may occur on admission or discharge from a clinical unit or hospital. Around 30% of these errors may cause patient harm. Such errors may occur when obtaining a list of medications a patient may be using or may have used. For example, a patient may take medication and forget to inform the hospital of the medication.

A medication module, as part of a mobile application (e.g., software on a smartphone), may enable the capture of medicine and usage of the medicine. The medication module may include a medication list and a medication diary. The mobile application may offer health portal connectivity (e.g., patients import medical records from their respective healthcare providers, including prescribed medication data). Patients may add, edit and/or remove (e.g., within the medication module) entries to the medication list (e.g., via RxNorm and openFDA application programming interfaces (APIs)). The medication diary may capture daily use of the medication (e.g., in real-time or retrospectively) based at least in part on one or more entries in the medication list.

The medication module may provide capabilities (e.g., robust capabilities) to help patients capture one or more (e.g., all) of the respective medications of the patients (e.g., both Rx's and OTCs). Patients may choose an entry of a medication based on one or more of a national drug code (NDC), active ingredient, or branded name. A text-based search may be assisted with one or more auto-prompt or auto-complete features (e.g., such that logging of medication may be minimally burdensome). A text-based search may be assisted with software that may offer a suggestion, a list, and/or other software to minimize the burden of entering medication. A medication may be entered directly and/or identified using, for example, optical character recognition (OCR) and/or dynamic scanning. Medications may be captured using database identifiers (e.g., RxCUIs) and/or medication identifiers (e.g., NDCs). In examples, a medication identifier may be linked to a database identifier (e.g., NDCs with linkage to RxCUI), which may facilitate sharing with healthcare providers (e.g., sharing the interoperability, aggregation, and/or analysis of medication utilization data for a research purpose).

Creating and maintaining a medication record may be challenging for patients and healthcare providers but may improve medication safety and robust real-world evidence generation. Rx and OTC medications and such respective usage may be captured (e.g., easily and robustly) via a medication module (e.g., through a patient-centric approach). Records within the medication module may be stored as standardized concepts (e.g., RxNorm and NDC) that may facilitate interoperability and/or aggregation. A mobile app may help (e.g., also help) to link medication usage with the clinical context and outcomes of such usage. In an example, patients may play a role (e.g., a central role) in the creation of a medication record for the patients.

The medication module may allow one or more profiles to be set up. A profile may be associated with a user. For example, one or more profiles may be set up to accommodate caregiving for children, elderly, and/or other dependents. The medication module may allow one or more profiles to be monitored. In an example, a profile for an adult may be monitored. In an example, a profile for a child may be monitored. In an example, a profile for the child may be monitored by a caregiver, which may be the adult.

The medication module may provide a user with the ability to capture both prescription and OTC medications. Medications may be entered via NDCs, active ingredients, or branded names. NDCs may be entered directly or identified by dynamic OCR without taking a static picture. Medication names may be prompted for confirmation.

In an example, text-based search may be assisted with auto-suggest and/or pick-list functionality, which may ensure that the logging of a medication is minimally burdensome. Medications may be unambiguously captured either as RxCUIs or NDCs (e.g., with linkage to RxCUI) that facilitate interoperability and aggregation.

In an example, the medication module may provide a system usability scale (SUS) survey. The SUS survey may be used to determine the ease and/or effectiveness of the medication module. For example, the SUS may be a 10-item scale to assess the medication module with statement of ease of use.

As disclosed herein, the medication module may include a medication list and a medication diary. The mobile app may offer health portal connectivity for patients to import medical records of the patients from the healthcare providers of the patients, including prescribed medication data to build a medication list. Patients may add, edit, and/or remove Rx and OTC entries to the medication list through integrations with the RxNorm and openFDA application programming interfaces (APIs). The patient may include pictures of a bottle (e.g., a prescription bottle) or packaging as part of the medication details. A picture may be displayed along with the name of the medication to help patients (e.g., a patient) identify the medication. A medication diary may capture the daily use of the medications (e.g., a quantity or unit) in real-time or retrospectively (e.g., based on entries in the medication list). A user (e.g., a patient) may edit and/or alter the mediation diary.

Semantic interoperability may present a challenge in using real-world data for evidence generation. Differences in source data captured using different standards and/or terminologies to represent semantically similar (e.g., equivalent) concepts may challenge a researcher's ability to reproduce real-world studies. Standardized terminological systems for biomedical information may benefit biomedical applications and research. Embodiments described herein may utilize standardized terminological systems for biomedical data.

Standardized terminological systems may include a standardized representation of a medicinal product, such as the Unique Ingredient Identifier (UNIT) thesaurus, the National Drug Code (NDC) coding system, the Systematized Nomenclature of Medicine-Clinical Terms (SNOMED CT) system, and/or the RxNorm system. In a standardized terminological system, medication information may include drug components such as trade name (if applicable), generic name, active ingredient(s), drug strength and unit of measure, dosage form, route of administration, chemical substance, drug class, mechanism of action, physiologic effect, manufacturer details, and package type and size.

Embodiments described herein may involve using RxNorm as a drug dictionary. RxNorm may be a hierarchical terminological system. For example, source data differences may be captured regionally and globally using different standards, terminologies, and exchange formats for the representation of the same or similar data elements. RxNorm may provide normalized names for clinical drugs, generic drugs, active ingredients, and/or the like. For example, drug names from RxNorm may be linked and/or mapped to drug vocabularies used in pharmacy management and/or drug interaction software (e.g., including those of First Databank, Micromedex, and Gold Standard Drug Database).

Standardized terminological systems for biomedical information have benefited biomedical applications and research. Practical use of biomedical information may (e.g., may often) request (e.g., require) mapping across terminological systems (e.g., the mapping may be a complex and time-consuming process). Embodiments described herein may use standardized terminology systems for biomedical information.

In examples, applications provided by the embodiments described herein may use standardized terminological systems for biomedical information. A standard nomenclature system, for example, may include medication information.

Medication information may include drug components such as a trade name (e.g., if any), a brand name, a generic name, an active ingredient(s), a drug strength, a unit of measure, a dosage form, a route of administration, a chemical substance, a drug class, a mechanism of action, a physiologic effect, a manufacturer details, a package type, a size, a combination thereof, and/or the like.

One or more terminological systems for the standardized representation of a medication may be used. These may include the Unique Ingredient Identifier (UNII) thesaurus, the National Drug Code (NDC) coding system, the Systematized Nomenclature of Medicine-Clinical Terms (SNOMED CT) system, the RxNorm system, a combination thereof, and/or the like. There may be differences in source data captured regionally and globally using different standards, terminologies, and exchange formats to represent the same or similar data elements. In examples, software may be provided to unify and/or standardize the drug information.

RxNorm is a hierarchical terminological system that provides normalized names for clinical drug entities codified through RxNorm Concept Unique Identifiers (RxCUIs). These concepts may be linked to one or more of the source drug vocabularies used. In examples, medicinal concepts covered by the RxNorm nomenclature may be used. Embodiments described herein may use RxNorm and/or term types (TTYs) to capture drugs or ingredients, such as an active ingredient, multiple active ingredients, a brand name, a branded drug, a branded drug component, a clinical drug, a clinical drug component, or the like. In examples, medicinal concepts within RxNorm may be represented through term types (TTY), with a TTY (e.g., each TTY) encapsulating a drug concept at a different level of granularity. A TTY may be assigned a RxCUI. For example, a clinical drug component (SCDC) TTY may consist of an ingredient coupled with the ingredient strength (e.g., “lisinopril 10 MG” [RxCUI 316151]); a clinical drug dose form (SCDF) TTY may consist of an ingredient coupled with a dose form (e.g., “lisinopril Oral Product” [RxCUI) 1164689]); and the like. Table 2 provides examples of TTY entities.

TABLE 2

| Term | | | |
|---|---|---|---|
| Type | | | |
| (TTY) | Description | Example | Example RxCUI |
| PIN | Precise ingredient | lisinopril anhydrous | 1546022 |
| IN | Ingredient | lisinopril | 29046 |
| SCDC | Semantic Clinical Drug | lisinopril 10 MG | 316151 |
| | Component | | |
| SCDF | Semantic Clinical Drug Form | lisinopril Oral Tablet | 372614 |
| SBDC | Semantic Branded Drug | lisinopril 10 MG | 567576 |
| | Component | [Prinivil] | |
| SCD | Semantic Clinical Drug | lisinopril 10 MG Oral | 314076 |
| | | Tablet | |
| SBD | Semantic Branded Drug | lisinopril 10 MG Oral | 206765 |
| | | Tablet [Prinivil]* | |
| SBDF | Semantic Branded Drug Form | lisinopril Oral Tablet | 368865 |
| | | [Prinivil] | |

Embodiments described herein may use and/or provide a standardized terminology dictionary and/or controlled terminology. With RxNorm being an ontological system, the TTY concepts in the system may be linked to one another via relations defined in the RxNorm ontology, which may allow for different levels of abstraction. As an example, the RxNorm clinical drug “lisinopril 10 MG Oral Tablet” [RxCUI 314076] “consists_of” the clinical drug component “lisinopril 10 MG”, which “has_ingredient” ingredient “lisinopril” [RxCUI 29046]. These relationships defined in the RxNorm ontology may be traversed to start with a specific TTY and achieve any level of desired abstraction.

In an example, branded drugs and clinical drugs may be selected as the basis for text-based medication entry. Branded drugs and clinical drugs may capture important medication details on ingredients, strength, and formulation, and may be consistent with medication data imported from EMR accounts (e.g., Epic or Cerner accounts), but also may distinguish between brand names and generic names. For example, users may enter either “Lipitor 40 MG Oral Tablet” (branded drug) or “atorvastatin 40 MG Oral Tablet” (clinical drug). In example, database hierarchies may be leveraged. For example, leveraging the RxNorm relationship and hierarchy, a branded drug may be mapped to a clinical drug. A clinical drug may be mapped to the clinical drug component, “atorvastatin 40 MG”, and further up to the ingredient “atorvastatin”.

In examples, branded drugs (e.g., SBD) and clinical drugs (e.g., SCD) may be selected as the basis for text-based medication entry. These two options may be consistent with the recommendation of the National Council for Prescription Drug Plans (NCPDP), where a drug product description may consist of three components: a drug name (s) (e.g., the medication ingredients or brand names), a medication strength(s), and a dosage form.

Embodiments described herein may use standardized and/or controlled terminology. Table 3 shows examples of a standardized terminology dictionary or controlled terminology. In an example, drugs may be recorded using NDC, GPI, or Multilex, while conditions may be documented as ICD-9, ICD-10, SNOMED, MedDRA, READ-OXMIS, or other local codes. In an example, related drugs and conditions (e.g., for certain analyses) may be aggregated to enable querying, searching, and analysis of observational data.

TABLE 3

BN—brand name
BPCK—branded pack
GPCK—generic pack
IN—ingredient
MIN—multiple ingredients
PIN—precise ingredient
SBD—branded drug
SBDC—branded drug component
SCD—clinical drug
SCDC—clinical drug component

Embodiments described herein may provide computer and/or mobile applications that may use National Library of Medicine (NLM) resources, including APIs such as RxNorm, RxTerms, prescribable RxNorm, RxClass, interactions, a combination thereof, and/or the like. Embodiments described herein may utilize medication information that is available online or locally. For example, embodiments may use an installable version of NLM resources, such as RxNav-in-a-Box.

In an example, the NLM may provide one or more. resources through an API such as RxTerms, MED-RT, and RxNorm. The API may be used by software described herein to leverage the NLM resources. For example, a software application may use the RxNorm API to facilitate the capture of medication data in the medication list. In an example, a free-text search of medications within the application may be assisted with an auto-suggest and pick-list function to populate the clinical drug or branded name concepts, including strength and formulation.

Embodiments described herein may leverage the RxNorm API to facilitate the capture of medication data in the medication list. In an example, a free-text search of medications within an application may be complemented with an autocomplete functionality where entries in the autocomplete list may be populated by RxNorm branded and clinical drug TTYs retrieved via the RxNorm API. The RxNorm API may be used to resolve a medication entered to a RxCUI. Table 4 shows an example of some of the RxNorm API resources that may be used.

TABLE 4

| | | |
|---|---|---|
| Function | Resource Endpoint | Endpoint Description |

Embodiments described herein may utilize one or more mechanisms for capturing drug data. In an example, drug data may be captured with an entry of a branded (e.g., SBD) or clinical drug component (e.g., SCD) TTY from RxNorm. Embodiments provided herein may capture drug data by using the entry of a Clinical Drug (SCD) concept from RxNorm, which may be used to address multi-ingredient products that may pose a challenge for users. The embodiments may provide a user with the ability to log a drug entry to their medication list unambiguously and allows for entry and logging of multi-ingredient products. For example, the RxNorm terminological set may have more than 22,000 SCD concepts. A search for clinical drug concepts containing the active ingredient “acetaminophen” [RxCUI 161] may return over 400 SCD terms, with approximately 89% being multi-ingredient concepts. It may be beneficial to capture multi-ingredient products as the public's general understanding of pharmacological ingredients may have a limited understanding of active ingredients in drug products. Embodiments herein may simplify searches by using a delimiter, such as the “/” delimiter, for the search of multi-ingredient products. For example, “acetaminophen/codeine” may be used as a search term for TTY concepts containing both “acetaminophen” and “codeine” ingredients. In examples, the absence of a “/” delimiter in a search may present SCD and SBD concepts containing a single ingredient.

In an example, a search for single-ingredient and a search for multi-ingredient drugs (e.g., single-ingredient vs. multi-ingredient drugs) may be performed at or near a similar time to improve performance (e.g., of RxNorm API calls). In an example, a search for single-ingredient and a search for multi-ingredient drugs (e.g., single-ingredient vs. multi-ingredient drugs) may be separated to improve performance (e.g., of RxNorm API calls). For example, a search of “acetaminophen” may return 440 possible entries (e.g., both single- and multi-ingredient) at a clinical drug level. Embodiments described herein may simplify the search such that a patient may more easily pick a drug from the entries (e.g., a list of the entries). In an example, the use of “/” may allow a user to search for multi-ingredient drugs when entering text, such as “acetaminophen/codeine.” In an example, embodiments may detect that “/” may not be used and may treat the search as a single ingredient search, reducing the number of single acetaminophen entries to 40.

The national drug code (NDC) may be a medication identifier and/or product identifier used in the United States for drugs intended for human use. The NDC allows the Food and Drug Administration (FDA) to maintain a list of drugs manufactured, prepared, propagated, compounded, and/or processed by the drug establishment for commercial distribution. Drug products may be identified and reported using a 10-digit, three-segment number. The 10-digit three-segment number may be called the National Drug Code (e.g., most often referred to as the NDC number). The three-segment number may be in a format, such 4-4-2 (e.g., 4 numbers, 4 numbers, 2 numbers), 5-3-2, 5-4-1, and the like. The FDA may assign the first segment, the labeler code. A labeler code may be the firm that manufactures or distributes the drug (e.g., including drug re-packagers or re-labelers). The labeler code may contain 4 or 5 digits. The second segment, the product code, may identify the drug (e.g., including the specific strength, dosage form, and formulation). The second segment code may contain 3 or 4 digits. The third segment, the product and package code (e.g., the product code), may identify package sizes and types. The third segment may contain 1 or 2 digits. The company may assign the third segment (e.g., the product and package code).

Drugs entering U.S. commercial distribution may be listed with the FDA. Companies may submit the NDC numbers assigned to the drugs manufactured or distributed by the firms (e.g., as part of the listing requirements for the firms). The FDA may publish the NDC numbers (e.g., the NDC numbers) and other information submitted by the labeler in an online NDC Directory or Database.

Under HIPAA-covered transactions, NDCs may be (e.g., need to be) reported in an 11-digit format. Government agencies and private payers may request (e.g., require) the NDC to be in an eleven (11) digit format and printed on patient instructions.

In an example, an 11-digit NDC (e.g., NDC11) may be used. For example, 11-digit NDCs may be used for the billing of drug products. The Centers for Medicare & Medicaid Services (CMS) and other government entities may use an 11-digit NDC (e.g., as part of their billing claim form). Government agencies, including HIPAA, may request the NDC in an 11-digit format with leading zeros.

An NDC may be mapped to RxNorm (e.g., many to 1). Multiple RxCUI returns may be present for a single NDC depending on the API calls.

Embodiments described herein may use OCR for medication identification. For example, OCR may be performed using one or more cloud based service, such as Amazon Textract, Google Vision AI, and/or Azure Computer Vision. OCR may be used to identify medication information, such as an NDC-formatted entity (e.g., 10 and/or 11 digits). OCR may be used to capture image-text; a brand name, and/or a generic name. For example, OCR may be used to capture data that may be used to make an API call that retrieves a brand name and/or a generic name from OpenFDA. OCR may be provided via a cloud-based service, a device-based service (e.g., dynamic), a combination thereof, and/or the like. A mobile-based OCR service (e.g., including Google ML) may be used in connection with OCR service for medication identification (e.g., NDC identification).

Cloud-based OCR service may include Amazon Web Service's Textract, Google's Vision AI service, and/or the like. In an example, the data extraction task may be performed server-side, and the user may be asked to submit one or more images to be processed via a web service request. If the event that text is not successfully captured from the server-side OCR solution, an additional image capture, web service request, and processing may occur.

In examples, an OCR service may be exposed through a software development kit (SDK) or a language-specific programming library that is capable of being deployed on-device. With the service being deployed locally on a client's device, these SDKs and libraries allow for interaction with the service in real-time, allowing for the service to be consumed without requesting a picture or network connection. The OCR service may include one or more mobile-based OCR services, such as Tesseract, Apple's Vision framework, Google's ML Kit Vision capabilities, a combination thereof, and/or the like.

In an example, the OCR service may be deployed on a smart device, such as both iOS and Android platforms. Compared with the text entry via RxNorm API calls, the use of NDCs, via dynamic OCR, may appear faster, easier, and more relatable. An OCR service may allow for improved capture and/or determination of drugs containing multiple ingredients.

Embodiments described herein may include feedback from pharmacists.

Embodiments described herein may provide instructions to help patients navigate the application, such as the medication list and medication diary.

Embodiments described herein may perform OCR by using a zoom feature to allow a user to focus on the text of a medication label.

Embodiments described herein may allow for a delay between when an image may be captured and when a scan of the image may be activated, for example, to reduce the impact of motion.

Embodiments described herein may perform multiple scans for labels with reflective backgrounds (e.g., a gold color) or when lower light conditions may exist.

Embodiments described herein may include in-app calculations of compliance and dosage.

In an example, an NDC number may be requested, but the NDC number may not be available on a drug label. Embodiments described herein may use information retrieved from the drug label to determine an NDC number. In an example, a universal product code (UPC) may be used for an OTC that may not have an NDC on the medication label. The UPC may be used to determine the NDC. The medication module may be used to determine text entries of names and/or NDC numbers.

OTC medicines marketed in the United States (e.g., a majority of the OTC medicines marketed in the United States) may carry an NDC number. For example, an OTC medication may have an NDC number on a box or a package. The availability of NDC numbers for prescription medicines may vary by repackaging and pharmacy leaflets.

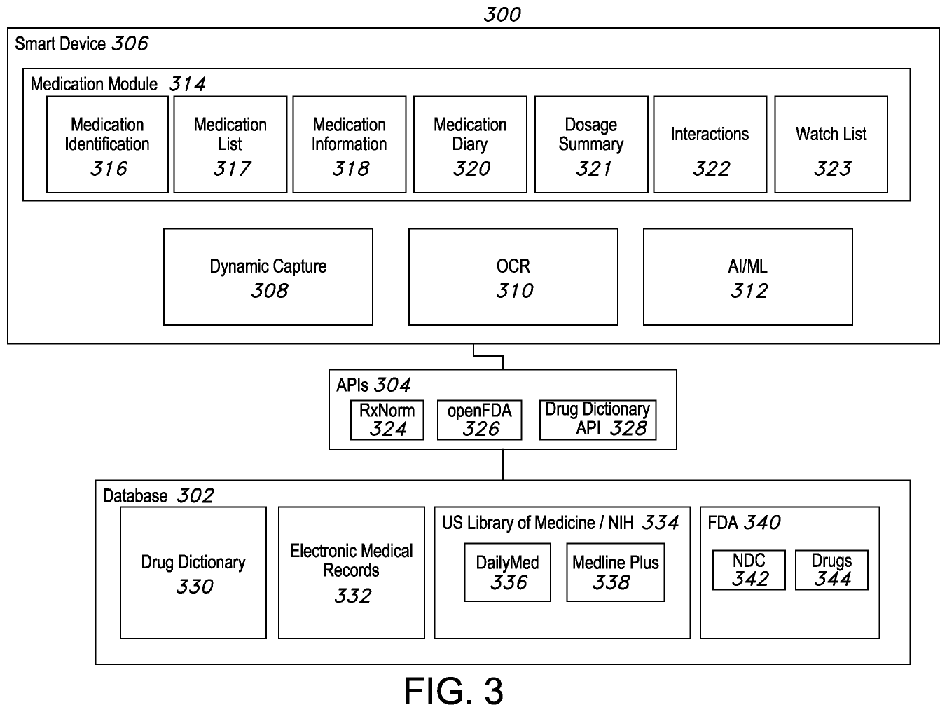

FIG. 3 depicts a block diagram of a system that may include one or more modules (e.g., software modules) for capturing medications and/or medication usage. As shown in FIG. 3, system 300 may include database 302, APIs 304, and smart device 306. Smart device 306 may be a smartphone, a tablet (e.g., an iPad), a smartwatch, a wearable device, a cellular phone, a computer, a server, and/or the like. For example, smart device 306 may be a smart device that may include components 120, shown with respect to FIG. 1. As another example, smart device 306 may be smartwatch 206, smartphone 204, or computing resource 212 shown with respect to FIGS. 2A-B.

Referring again to FIG. 3, smart device 306 may comprise one or more software modules, such as dynamic capture 308, optical character recognition (OCR) 310, artificial intelligence/machine learning (AI/ML) 312, and/or medication module 314.

Medication module 314 may include medication identification 316, medication list 317, medication information 318, medication diary 320, dosage summary 321, interactions 332, and/or watch list 323. Medication identification 316 may allow smart device 306 to determine an identity of a medication. For example, medication identification 316 may receive an image and/or video. Medication identification 316 may request OCR 310 to process the image and/or video to retrieve text from the image and/or video. The text may be used by medication identification 316 to determine a medication identifier, which may include one or more of an identification number (e.g., an NDC), an active ingredient (e.g., a generic drug name, or a brand name.

As described herein, a user may use medication module 314 to capture medication and/or medication usage. In an example, medication identification 316 may assist a user in identifying a medication. Medication list 317 may determine a list of medications that a user is using. And the list of medication may include the identified medication. For example, the identified medication may be added to a list of medications by medication list 317. Medication list 317 may allow a user to capture a photo and assign that photo to the identified medication. Medication information 318 may provide medication information for the identified medication. Medication diary 320 may record when a user consumed the identified medication. And dosage summary 321 may determine and/or display a summary of how much of an active ingredient a user consumed, where the identified medication may comprise the active ingredient.

As another example, medication identification 316 may receive data from a user (e.g., a patient), such as a text entry. The text may indicate the identity of a medication. The text may indicate an NDC, and medication identification 316 may perform a search and/or verification of the NDC using APIs 304. The text may indicate an active ingredient for the medication, and medication identification 316 may determine a medication identifier associated with the active ingredient. The text may indicate a brand name (e.g., a manufacturing name, a store name, and/or the like). Medication identification 316 may determine a medication identifier associated with the brand name.

Medication identification 316 may verify the medication identifier by using APIs 304. For example, medication identification 316 may use the RxNorm API to send a request to verify an NDC. Medication identification 316 may use APIs 304 to send a request for information regarding an identified medication. For example, medication identification 316 may use open FDA to send a request for information regarding a medication identified with a RxCUI and/or an NDC.

Medication identification 316 may determine whether a medication comprises one or more active ingredients. For example, many medications, such as cold medications, include one or more active ingredients that may be used to address patient symptoms. Medication identification 316 may identify one or more of the active ingredients and may provide a list of those ingredients to a user. Medication identification 316 may identify one or more active ingredients and provide the ingredients to another module, such as medication list 317, medication information 318, medication diary and/or summary 321.

In an example, medication identification 316 may allow a user to perform a free-text search of medications. Medication identification 316 may provide an autocomplete functionality where entries in the autocomplete list may be populated by RxNorm branded and/or clinical drug TTYs retrieved via the RxNorm API. The RxNorm API may be used to resolve a medication entered by a user using a RxCUI. Medication list 317 may provide a list of medications that a user, such as a patient, may be using. For example, medication list 317 may provide a list of one or more medications a user has consumed. Medication list 317 may include or may be a self-reported list. For example, the user may use device 306 to record a medication they are using such that medication list 317 may provide a list of current medications used by the user.

Medication list 317 may allow a user, such as a patient, to add, edit, and/or remove prescription (e.g., Rx) and OTC entries to a medication list. Medication list 317 may include integrations with one or more APIs, such as RxNorm and openFDA, which may allow medication list 317 to identify a medication provided by a user. The user may include pictures of a bottle (e.g., a prescription bottle) or packaging and/or actual medications as part of the medication details. Medication list 317 may store this picture such that the picture may be displayed along with a name of the medication to help a user (e.g., a patient) identify the medication.

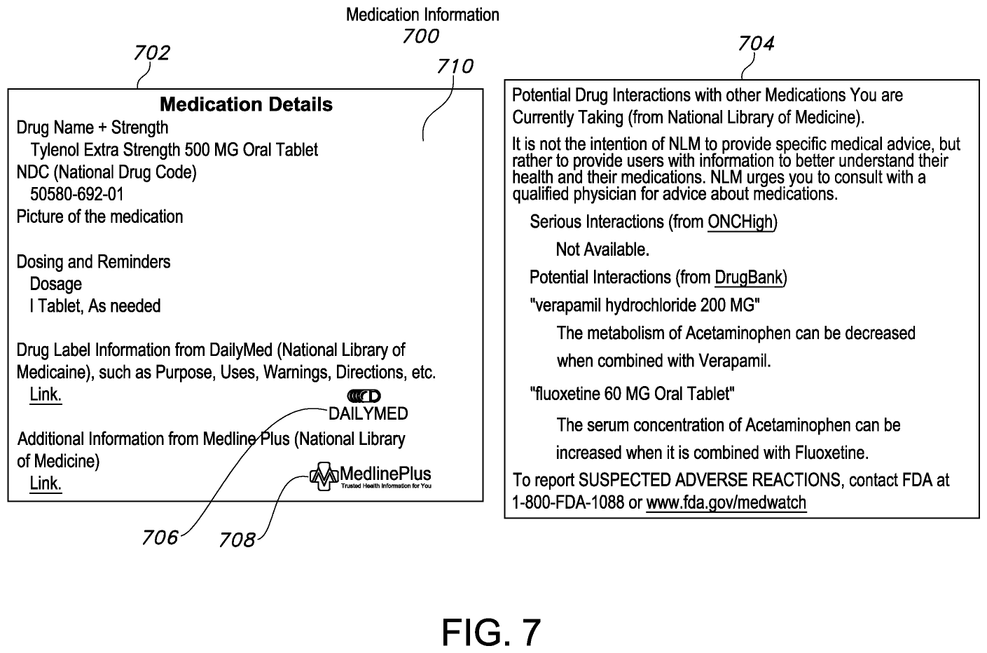

Medication information 318 may provide a user with information about medication. An example of the medication information may be seen at 700 in FIG. 7.

Referring again to FIG. 3, the medication information 318 module may provide medication information that may include details regarding the medication, such as the drug name and strength (e.g., dosage). For example, the user may be informed that the identity of the medication is Tylenol extra strength 500 mg oral tablets. The medication information may include an NDC code. For example, the user may be informed that the NDC code is a 10 or 11-digit format in NDC and may be provided with the NDC. The medication information may include a picture of the medication from the manufacturer, FDA, regulatory authority, combination thereof, and/or the like. The medication information may include instructions, dosing, and/or reminders. For example, the user may be informed as to how the medication may be used. The medication information may include links and/or information from an external source, such as from DailyMed at MedlinePlus.

Embodiments described herein may provide medication reminders. For example, a user may be provided with a daily reminder to record their medication. The reminders may be specific to one or more medications. For example, a first reminder may be set and/or provided for a first medication, and a second reminder may be set and/or provided for a second medication. In an example, a reminder may be provided for a group of medications (e.g., all medications).

The medication information may include potential drug interactions with the user's other medications. The warnings regarding potential drug interactions may be received and/or determined from the National Library of Medicine, the FDA, a regulatory authority, and/or the like.

Medication diary 320 may allow a user to enter the medication they have consumed along with the time and/or date they consumed the medication. Medication diary 320 may log when a user may have consumed the medication along with the identity of the medication. Medication diary 320 may use APIs 304 to retrieve data from database 302 regarding medications the user may have previously consumed, dates when those medications had been consumed, times when those medications had been consumed, and/or the like. Medication diary 320 may capture the daily use of the medications (e.g., a quantity or unit) in real-time or retrospectively (e.g., based on entries in the medication list 317).

Dosage summary 321 may provide smart device 306 with the ability to determine how much medication and/or how much of an activity ingredient a user may have consumed within a period. For example, dosage summary 321 may determine an hourly dosage (e.g., 8 hours, 12 hours, 23, hours, 48 hours, etc.), a daily dosage, a weekly dosage, a monthly dosage, a combination thereof, and/or the like. The dosage may be for an active ingredient. For example, dosage summary 321 may determine a daily dosage for acetaminophen.

Dosage summary 321 may determine how many active ingredients a user may have consumed and how many of those active ingredients the user may have consumed. For example, if the user consumes a cold medication with three active ingredients and a headache medication with two active ingredients, dosage summary 321 may provide a summary of the five active ingredients.

As another example, the user may consume a first medication that includes 100 mg of caffeine, 500 mg of aspirin, and 500 mg of acetaminophen. The user may consume a second medication that includes 600 mg of acetaminophen and 30 mg of codeine. Medication identification 316 may identify the first medication, the second medication, and the active ingredients of the first medication and the second medication. Dosage summary 321 may summarize the active ingredients such that the user may be notified that the user consumed 100 milligrams of caffeine, 500 mg of aspirin, 1100 mg of acetaminophen, and 30 mg of codeine.

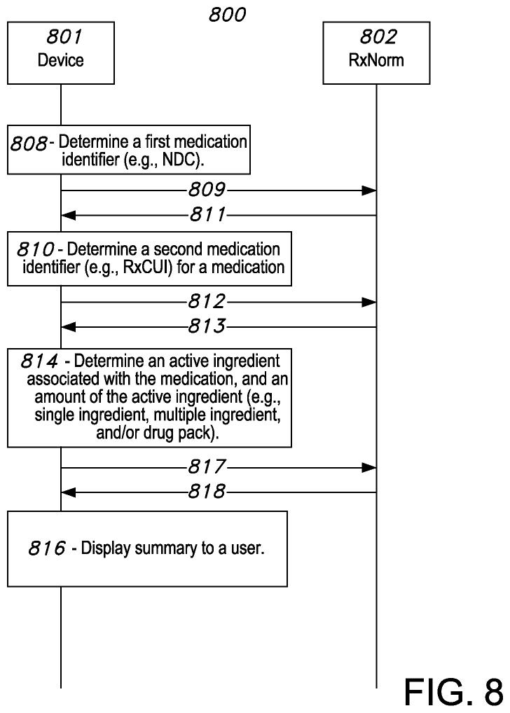

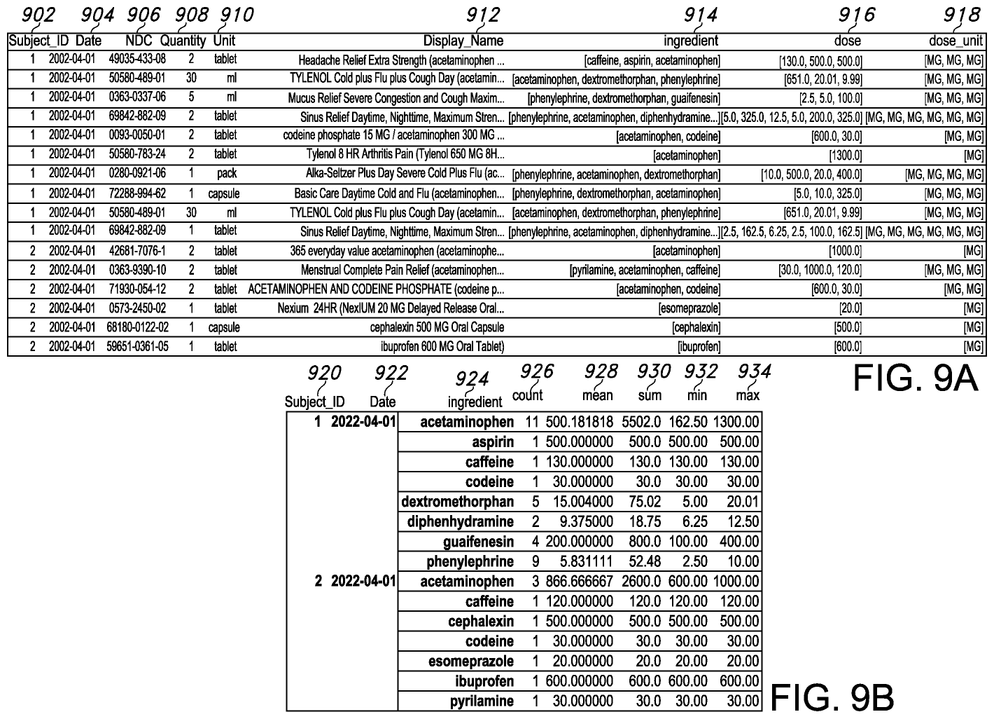

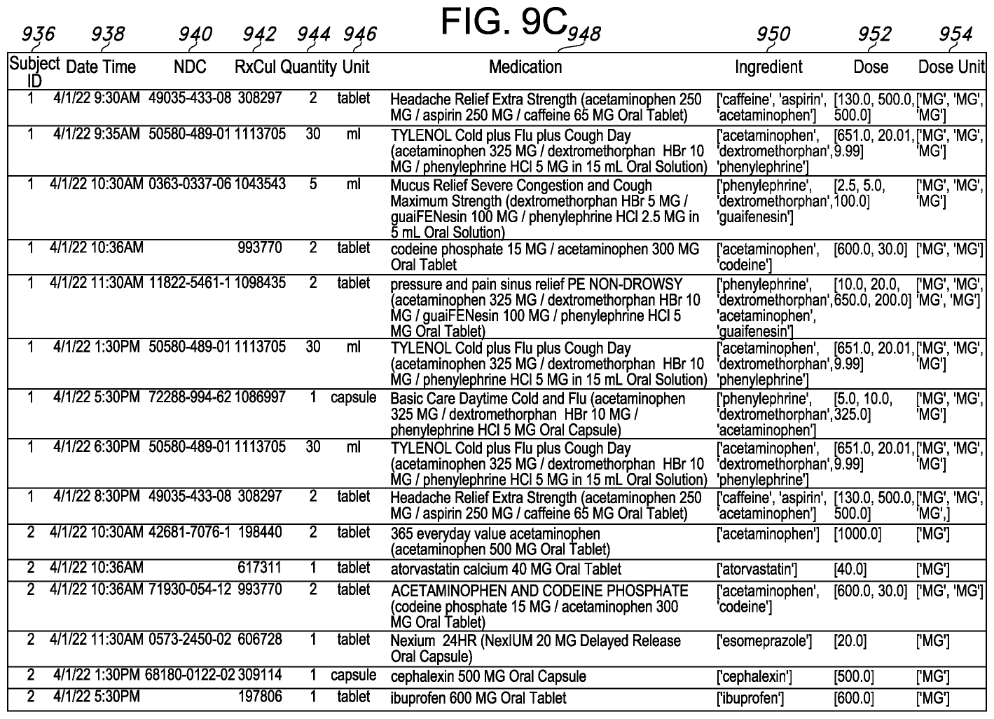

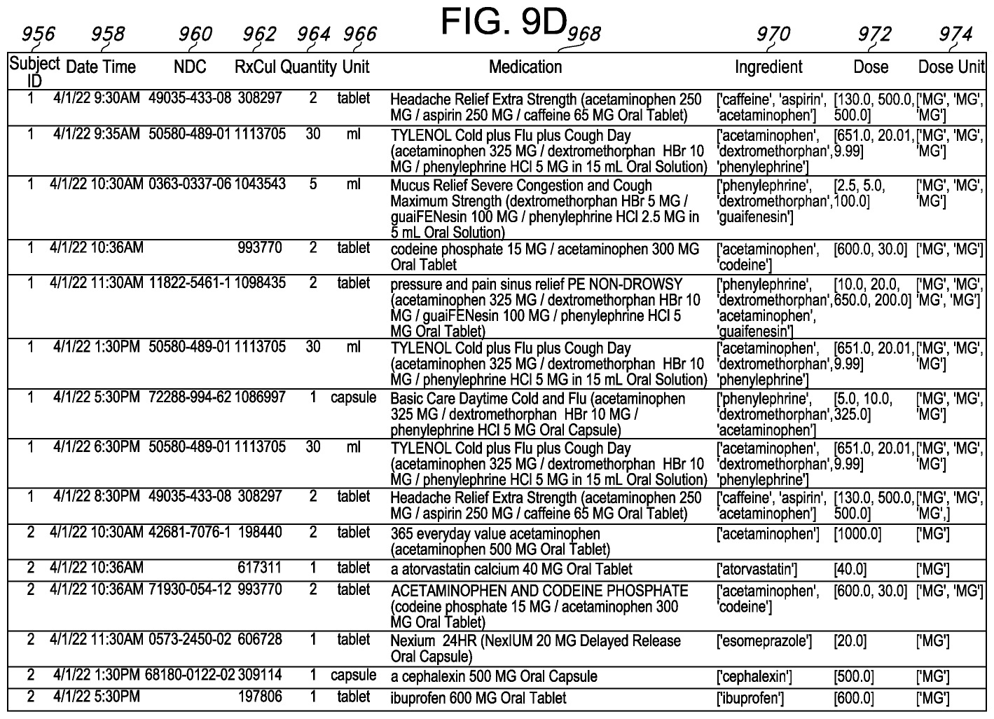

Dosage summary 321 may include information for one or more medications. For example, dosage summary 321 may, in association or communication with medication information 318, include information for one or more medications. The information may include a name of a medication, an NDC, an image and/or video of the medication, label information, a link to label information, ingredient information, a link to ingredient information, drug-to-drug interaction, adverse effect reporting, a combination thereof, and/or the like. Dosage summary 321 may, for example in association or communication with medication information 318, perform the method shown with respect to FIG. 8 and/or may provide the information shown with respect to FIGS. 9A-C.

Referring again to FIG. 3, interactions 322 may analyze one or more medications that have been or may be consumed by a user. Interactions 322 may determine if an interaction between the medications might transpire. The interaction may include interactions between medications, interactions between a medication and a food, interactions between a substance (e.g., alcohol, illicit drugs, legal drugs, etc.) and a medication, a combination thereof, and/or the like.

Interactions, such as medication interactions, may be categorized into several types, for example depending on their effects on the patient's health.

A type of medication interaction may be a synergistic effect, which may occur when two or more drugs are taken together, and their combined effects are greater than the sum of their individual effects. For example, some combinations of medications may enhance the overall therapeutic effect, potentially leading to more effective treatment of a health condition.

A type of medication interaction may be an adverse drug interaction, which may take place when the combination of medications results in a harmful effect. These may include increased risk of side effects, decreased effectiveness of one or both drugs, and/or creation of a health issue not present before the drugs were combined.

A type of medication interaction may be an antagonistic interaction, which may occur when one drug reduces the effectiveness of another. For example, one or more antibiotics may interfere with the absorption of birth control pills, potentially reducing their effectiveness.

A type of medication interaction may be a pharmacokinetic interaction. This may occur when one drug affects the body's ability to process another medication, perhaps altering its absorption, distribution, metabolism, and/or excretion.

Interactions 322 may monitor one or more medications that a user may consume and may evaluate the possible interactions between these medications. Interactions 322 may anticipate and/or prevent drug interactions that may occur. For example, interactions 322 may determine that a first medication that a user consumed may have an interaction with a second medication that a user consumed.

Interactions 322 may notify a user about interactions and/or possible interactions. In an example, interactions 3222 may generate alerts or reminders, potentially enabling a user to adjust their medication intake or consult with healthcare professionals.

Interactions 322 may learn from the user's medication history. For example, over a period of time, interactions 322 may identify patterns or trends, which may assist in refining its interaction analysis and prediction capabilities.

Watch list 323 may supervise a list of medications provided by a healthcare provider (HCP) The HCP may be a pharmacist, a doctor, a surgeon, and/or the like. In an example, a doctor may wish to ensure that a patient is avoiding a specific class of drugs that may interact with a medication that the doctor has prescribed for the patient.

Watch list 323 may monitor medication, may maintain a record of medications that pose a risk to the patient due to potential interactions with other drugs, a combination thereof, and/or the like. Watch list 323 may provide a safeguard for the patient by alerting them or their healthcare provider about potential risks.

In an example, a surgeon may want to ensure that a patient avoids one or more medications prior to surgery. Watch list 323 may function by alerting the surgeon or patient if a potentially harmful medication is detected in the patient's list of current medications.

Watch list 323 may receive updates from the HCP or the HCP's system about changes in the patient's medication regimen. Watch list 323 may receive an update regarding research pertaining to drug interactions. This may help keep the watch list up-to-date and increase the accuracy of its monitoring function.

Watch list 323 may allow for manual input from healthcare providers or patients themselves. This may provide a layer of customization that may allow for the inclusion of one or more medications that may be relevant for a patient due to their health status or condition.

Watch list 323 may provide an interface that allows users to view and understand the information it contains. This may include a list of medications to avoid, reasons for their inclusion in the list, and possible consequences of ignoring these warnings. Dynamic capture 308 may be used to capture an image, a video, and/or the like. Dynamic capture 308 may also provide image and/or video processing. For example, smart device 306 may use dynamic capture 308 to capture an image of a medication label, which may be used to determine the identity of the medication and/or other information about the medication. As another example, dynamic capture 308 may allow a user to point a camera of smart device 306 at a label to capture information regarding the medication without having the user record an image or video.

OCR 310 may determine text and/or other information from an image or video. For example, OCR 310 may be used to determine the text of a label. The text may be used to determine information regarding the medication. The text may include a national drug code (NDC). For example, OCR 310 may detect an NDC, active ingredient, a brand name, a combination thereof, and/or the like. The detected information may be used by smart device 306 to determine the identity of a medication and/or an active ingredient associated with the medication.

AI/ML 312 module may be used to analyze and/or capture personalized medication data, medication usage data, medication consumption data, and medication data associated with a user (e.g., a patient). For example, an image or a video of a medication label may be analyzed by AI/ML 312 to determine the identity of the medication. As another example, an image or a video of a medication may be used to determine an NDC associated with the medication and may be used to determine an active ingredient that is included in the medication.

AI/ML 312 may include machine learning, a branch of artificial intelligence that seeks to build computer systems that may learn from data without human intervention. These techniques may rely on creating analytical models that may be trained to recognize patterns within a dataset, such as a collection of medication labels, medication images, and/or the like. These models may be deployed to apply these patterns to data, such as medication labels, to improve medication identification. AI/ML 312 may include supervised machine learning, unsupervised machine learning, reinforcement learning, and/or cognitive computing (CC). For example, AI/ML 312 may use cognitive computing (CC) to utilize one or more self-teaching algorithms that may use data mining, visual recognition, and/or natural language processing to identify medications and/or active ingredients associated with the medications.

APIs 304 may allow smart device 306 to send and/or retrieve data from database 302. For example, smart device 306 may use APIs 304 to retrieve data about medications from a drug dictionary, electronic medical records (EMR), the US library of medicine, the National Institute of Health (NIH), the Federal Drug Administration (FDA), a combination thereof, and/or the like. Although APIs 304 may be shown as including RxNorm, openFDA, and Drug Dictionary API, APIs 304 may include additional APIs that may be provided by regulatory agencies, private companies, pharmaceutical companies, hospitals, EMR providers, machine learning providers, cloud software providers, a combination thereof, and/or the like. For example, Drug Dictionary API may be an API that may allow device 306 to send and/or receive data from a commercial drug dictionary, such as REDBOOK and/or the Gold Standard Drug Database.

APIs 304 may include RxNorm. RxNorm is an API that the National Library of Medicine provides. RxNorm may use and/or may provide a unified medical language system. RxNorm may provide normalized names for clinical drugs and may link drug names to many of the drug vocabularies used in pharmacy management and drug interaction software, including those of First Databank, Micromedex, and the Gold Standard Drug Database. By providing links between these vocabularies, RxNorm may mediate messages between systems not using the same software and vocabulary. RxNorm may allow access to data and/or databases that use United States Pharmacopeia (USP) Compendial Nomenclature from the United States Pharmacopeial Convention. For example, RxNorm may allow for access to a cumulative data set of Active Pharmaceutical Ingredients that USP may provide.

In an example, RxNorm may provide a brand name, a generic name, and/or an active ingredient for a medication or drug. The brand name recognized by RxNorm may be analogous to a trademark name, such as Zocor or Lipitor, which may be first to market drugs. Products that follow the first to market drugs (e.g., when patent protection for those drugs may be over) may be referred to as generic drugs. For example, a drug with an active ingredient equivalent to a brand name drug may be referred to as a generic drug.

APIs 304 may include openFDA. OpenFDA is an Elasticsearch-based API provided by the FDA that may include (e.g., serves) public FDA data about drugs, devices, and foods. OpenFDA may allow access to data regarding recall enforcement reports, adverse events, drug information, drug reaction information, a combination thereof, and/or the like. OpenFDA may allow for access to one or more databases that the FDA provides.

OpenFDA may provide a brand name and/or a generic name for a medication or drug. A brand name may be a name provided by a store or manufacturer, such as “up and up allergy relief” from Target. A generic drug may be a pharmaceutical drug or medication containing the chemical substance or active ingredient of a medication originally protected by a patent. OpenFDA may be used to retrieve product-level details, which may include the structured product label (SPL) identification (ID) for the product. The product-level details may be used to identify a product.

Database 302 may include medical data, medication data, data regarding the identification of a medication, data regarding the effects of medication, data regarding side effects of the medication, patient information, prescriptions, doctor instructions, a combination thereof, and/or the like. Database 302 may include private, public, commercial, regulatory, and/or similar databases. For example, database 302 may include data from an Apple Watch, iCloud, or the like. Although the databases of regulatory agencies based in the United States are shown with respect to FIG. 3, the databases of other regulatory agencies outside the US may be used. Database 302 is not limited to the database shown with respect to FIG. 3, and other databases which may have data regarding medication and/or the identity of medications may be used.

Database 302 may include or may include data from a drug dictionary, an EMR, a government agency, a health agency, a regulatory agency, a hospital, a research institution, a consumer database, a combination thereof, and/or the like. A drug dictionary may include information regarding one or more medications (e.g., drugs). For example, a drug dictionary may include information regarding an NDC associated with a medication, a RxCUI associated with medication, an active ingredient associated with a medication, a brand name, an OTC name, a generic name, a prescribable synonym, a combination thereof, and/or the like. A drug dictionary may be public or private. For example, a drug dictionary may be a commercial drug dictionary such as REDBOOK and/or the Gold Standard Drug Database.

Although database 302 may be shown as incorporating data and/or a database from the US Library of Medicine, the National Institute of Health (NIH), and the Federal Drug Administration (FDA), other databases and/or regulatory agencies (e.g., regulatory databases) may be used. For example, the embodiments described herein may use medical identifiers provided by a country such as those shown in Table 1.

As shown in FIG. 3, database 302 may include data from or may be a database provided by the US Library of Medicine (NLM) and/or the National Institute of Health (NIH), such as DailyMed and Medline Plus. Database 302 may include data from or may be a database provided by the FDA, such as an NDC database and a registered drug database.

Database 302 may include data from or may include DailyMed. DailyMed is a website and/or database that the NLM operates to publish drug labels (which may be referred to as a packet insert). The contents of DailyMed may be updated by the FDA using information that may be received from the pharmaceutical industry (e.g., information obtained from manufacturers).

Database 302 may include data from or may include MedlinePlus. MedlinePlus is an online information service produced by the United States National Library of Medicine. MedlinePlus provides information from the National Library of Medicine (NLM), the National Institutes of Health (NIH), other U.S. government agencies, and health-related organizations. MedlinePlus provides encyclopedic information on health and drug issues and a directory of medical services. MedlinePlus may provide information on medications.

Database 302 may include data from or may include a drug database provided by the FDA. The drug database may include the identities of drug products that may be approved based on safety and effectiveness by the FDA (e.g., the orange book). The drug database may include data regarding therapeutic equivalence evaluations for prescription drugs, such as generic drugs. The drug database may include prescription brand-name drug products, generic drug products, therapeutic biological products, over the counter (OTC) brand-name products, OTC generic drugs, a combination thereof, and/or the like. The drug database may include complementary medicines such as vitamins, herbal medicines, and/or alternative therapies. The drug database may include patient information, product labels, approval letters regarding drugs, reviews of drugs, and/or other drug-related information.

Database 302 may include data from or may include the NDC database. The NDC database may track products (e.g., at a granular level), including the active ingredient, strength, package, manufacturer, and/or adverse events. An adverse event may be related to an active ingredient with a particular strength (e.g., Vioxx 50 mg) isolated to individual manufacturers due to excipients, quality control, or contamination.

The NDC database provides for medications to be listed according to a national drug code (NDC). A national drug code (NDC) is a product identifier used in the United States for drugs intended for human use. For example, the Drug Listing Act of 1972 ensures that registered drug establishments provide the Food and Drug Administration (FDA) with a list of drugs manufactured, prepared, propagated, compounded, or processed for commercial distribution. Drug products may be identified and reported using the NDC. Although embodiments shown herein may use an NDC, other medication identifiers such as Generic Product Identifiers (GPI) may be used.

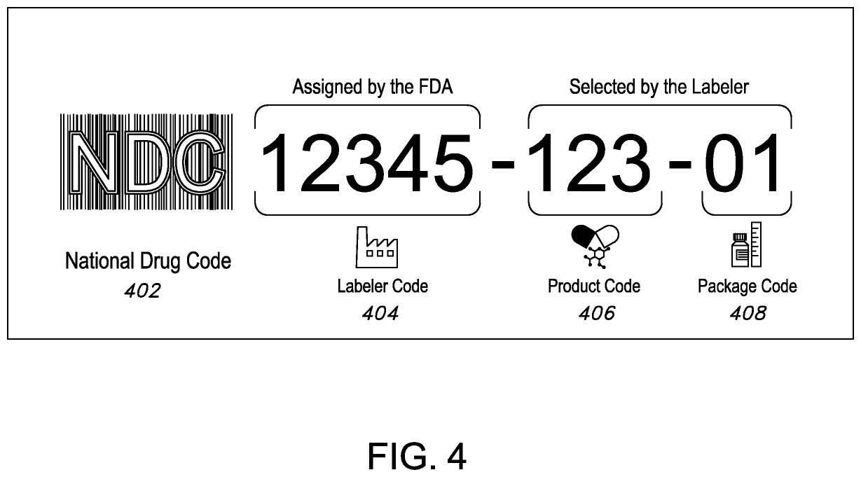

FIG. 4 depicts an example format for a National Drug Code (NDC). An NDC may be in a format. For example, an NDC may be in a 9-digit format, a 10-digit format, an 11-digit format, or a 12-digit format. Although embodiments described herein may be described with regard to 9-digit, 10-digit, 11-digit, and/or 12 w digit formats, other formats may be used, such as formats that comprise a variety of digits, characters, and/or lengths.

The format of the NDC may comprise one or more portions. The format may comprise at least one of a labeler code, a product code, or a package code. For example, the format of the NDC may comprise a first portion, which may be a labeler code, and a second portion which may be a product code. As another example, the format of the NDC may comprise a first portion, which may be a labeler code, a second portion, which may be a product code, and a third portion, which may be a package code.

As shown in FIG. 4, NDC 402 may be a 3-segment numeric identifier and may be assigned to a medication listed under the US Federal Food, Drug, and Cosmetic Act. The segments may identify the labeler or vendor, product, and/or the trade package (e.g., of this product).

Labeler code 404 may be assigned by the Food and Drug Administration (FDA) upon a Labeler Code Request submission. A labeler may be any firm that manufactures, repacks, or distributes a drug product. Labeler code 404 may be 4, 5, or 6 digits long.

Product code 406 may be 3 or 4 digits long, and may identify a firm's specific strength, dosage form, and formulation.

Package code 408 may be 1 or 2 digits long and may identify package forms and sizes. In some examples, product and package segments may have contained characters other than digits.

While the FDA may assign the labeler code, both the product and package segments may be assigned by the labeler. In the past, labelers may have had the opportunity to reassign old product codes no longer used to new products. Currently, under the FDA validation procedures, when an NDC code is assigned to a product (e.g., defined by key properties, including active ingredients, strength, and dosage form), it may not be later reassigned to a different product.

NDC codes may exist in one or more groupings, such as the following groupings of digits into segments: 5-4, 4-4-2, 5-3-2, 5-4-1, 6-3-2, or 6-4-1. For example, an NDC code may be in a 9-digit format that may include a labeler and product code grouping. For example, NDC 402 may be comprised of Labeler Code 404 and Product Code 405 (5-4), which may be used to identify a product regardless of its packaging.

In examples, the NDC structure rules may depend on the length of certain segments. The following rules may apply to the 10 or 11-digit format:
If a labeler code may be 5 or 6 digits in length, it may be combined with the following
   A product code consisting of 4 digits and a package code consisting of 1 digit for a total NDC length of 10 or 11 digits (5-4-1 or 6-4-1), or
   A product code consisting of 3 digits and a package code consisting of 2 digits for a total NDC length of 10 or 11 digits (5-3-2 or 6-3-2).
If a labeler code is 4 digits in length, it may be combined with a product code consisting of 4 digits and a package code consisting of 2 digits for a total NDC length of 10 digits (4-4-2).
A registrant or private label distributor with a given labeler code may use a Product-Package Code configuration (e.g., a 3-digit product code combined with a 2-digit package code, or a 4-digit product code combined with a 1-digit package code).

An NDC may be printed on a box and/or bottle of medication, which may be a prescription, and OTC, and/or the like. For prescriptions that may be re-packaged without the original packing, the NDCs may be available on the medication instructions that may be disturbed to patients. In examples, an NDC may not appear on a drug label, and embodiments described herein may take one or more actions to identify the medication and/or NDC.

An NDC may be a source NDC, a package NDC, and/or a product NDC. A source NDC may be an NDC determined from a product label and/or medication information. A package NDC may be an NDC in a 10-digit or 11-digit format. A package NDC may comprise a labeler code, a product code, and a package code. A product NDC may be in a 9-digit format. A product NDC may comprise a labeler code and a product code. Although digits are referred to with respect to an NDC, an NDC may also be in a character format such as a 9, 10, 11, or 12-character format.

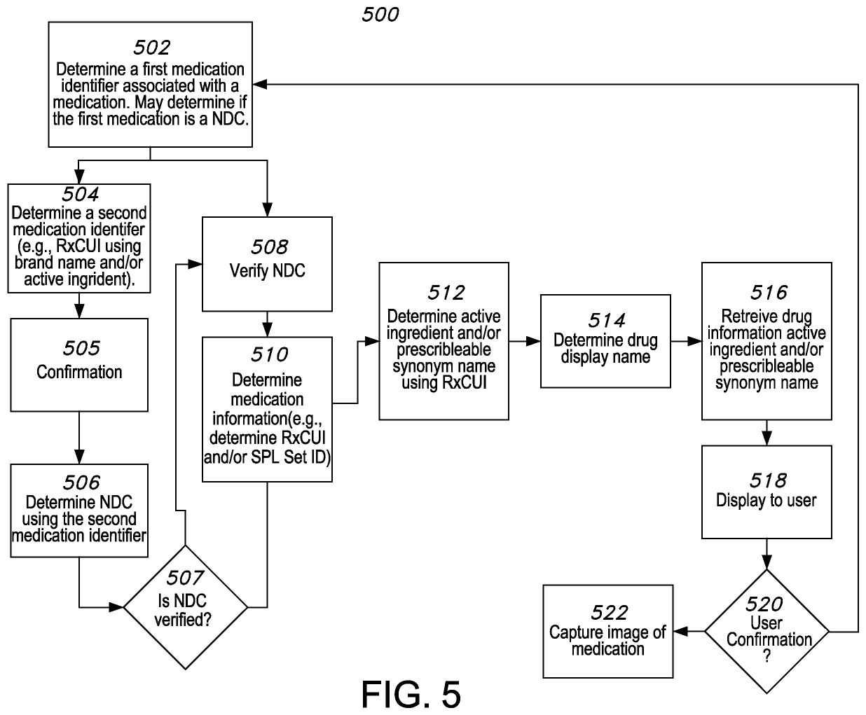

FIG. 5 depicts an example method for capturing medications and/or medication usage. A medication may be an over the counter (OTC) medication, a prescription medication, a branded medication, a generic medication, a combination thereof, or the like. As shown in FIG. 5, method 500 may perform one or more of 502, 504, 505, 506, 508, 510, 512, 514, 516, 518, 520, and 522 to capture a medication and/or a medication usage. Method 500 may be performed by a smart device, which may comprise one or more of the components shown in FIG. 1.

Referring again to FIG. 5, at 502, a first medication identifier associated with a medication may be determined. In identifying the medication, it may be determined whether the medication is a prescription or over-the-counter medication. In an example, different methods may be performed to determine the first medication identifier based on whether the medication may be a prescription or an over-the-counter medication.

The first medication identifier may be an NDC, a regulatory identifier, a branded name, a generic name, an active ingredient, and/or the like. In examples, the active ingredient may be the generic name or vice versa. The first medication identifier may be a source NDC.

In an example, at 502, it may be determined if an NDC associated with a medication is available. A user may use a smart device camera to take an image and/or video of the medication. The smart device may determine whether an NDC is included within the image and/or video of the medication. The smart device may determine that an NDC may not be included within the image and/or video.

If the smart device determines that an NDC is available, it may try to verify the NDC at 508. The NDC may be determined to be a source NDC. For example, if the NDC is verified, the NDC may be assigned as a source NDC. If the smart device determines that an NDC is unavailable, it may try to determine a second medication identifier at 504.

At 504, a second medication identifier may be determined. The second medication identifier may be an NDC, a regulatory identifier, a RxCUI, a brand-name, a generic name, and/or active ingredient. The second medication identifier may be determined by receiving a description of the medication from a user and performing a search for the medication using a database such as RxNorm. In an example, a user may enter a description of the medication, such as the brand name, generic name, and/or active ingredient of the medication. The smart device may send a query to a database using an API, such as RxNorm, and may receive a database identifier for the medication, such as a concept unique identifier (RxCUI), a structured product label identifier (SPL ID), a combination thereof, and/or the like. The database identifier may be assigned as the second medication identifier.

At 505, a confirmation may be performed. For example, the device may request that the user confirm the second medication identifier. Embodiments described herein may leverage the RxNorm API to capture medication data in the medication list.

A user may provide a medication identifier for a medication. In an example, a user may type in an NDC. In an example, an NDC may not be available to the user and the user may provide a name for the medication. A free-text search of medications within an application may be complemented with an autocomplete and/or confirmation functionality. For example, where entries in an autocomplete, a list may be populated by RxNorm branded drug and clinical drug TTYs retrieved via the RxNorm API. The RxNorm API may be used to resolve a medication entered by a user using a RxCUI.

At 506, one or more databases may be queried using the second medication identifier, and the query results may, for example, map the second medication identifier to an associated NDC. In an example, an associated NDC may be mapped to the second medication identifier, and the associated NDC may be assigned as the first medication identifier. In an example, an NDC may be found, and additional database identifiers (e.g., an SPL ID) may be determined.

At 507 it may be determined if the NDC is verified. If the NDC is not verified, then the NDC may be verified at 508. If the NDC is verified, then the NDC may be used at 510 to retrieve additional information and/or identifiers (e.g., an SPL ID).

At 508, an NDC may be verified. For example, if the first medication identifier indicates that it is NDC, that NDC may be verified against a regulatory database, such as the NDC database provided by the FDA. This may be done, for example, to verify an NDC determined from a medication label to ensure accurate detection of the medication. In examples, an NDC to be verified may be translated to one or more formats to allow verification against one or more databases.

At 510, medication information associated with medication may be determined, for example, using a first medication identifier and/or a second medication identifier. For example, the first medication identifier and/or the second medication identifier may be used to retrieve data from one or more databases. The data from one or more databases may be combined. In examples, the first medication identifier and/or the second medication identifier may be used to determine a database identifier, such as a RxCUI, and a product identifier, such as a SPL ID.

At 512, one or more active ingredients associated with the medication may be determined. In an example, the first medication identifier and/or the second medication identifier may be used to query one or more databases to determine active ingredients and/or prescribable synonyms for the medication. In an example, a prescribable synonym for the medication may be a generic name for the medication.

In an example, the active ingredient may be associated with a prescription medication, an OTC medication, a complementary medication, a combination thereof, and/or the like. The complementary medication may include vitamins, herbal medicines, and/or alternative therapies.

A medication may be comprised of one or more active ingredients. For example, a cold medication may include three active ingredients. By using the first medication identifier and/or the second medication identifier, the active ingredients may be identified, and information regarding those ingredients may be determined. For example, warnings, recalls, patient information, side effect information, dosage information, strength of the medication, interactions with other medication, and/or the like may be retrieved.

At 514, a drug display name may be determined. The drug display name may be a combination of a brand name and/or one or more active ingredients. For example, the drug display name may be “Tylenol (acetaminophen).” The drug display name may be determined to indicate the active ingredients, the dosage of active ingredients, a generic medication name, a combination thereof, and/or the like. For example, the drug display name may be determined to clarify what active ingredients a patient may be consuming.

The drug display name may be selected to prevent confusion to a user. The drug display name may be selected to indicate a common name that may be known to a user. For example, the display name may indicate that the medication is similar or equivalent to a brand name, such as Tylenol.

At 516, drug information regarding active ingredients and/or prescribable synonym names may be determined. For example, the medication may be an over-the-counter medication comprised of one or more active ingredients. The prescribable synonym for the over-the-counter medication may be determined. For example, acetaminophen medicine may be determined for the over-the-counter medication Tylenol. In an example, it may be determined that the medication is an over-the-counter medication that is comprised of one or more active ingredients, such as a cold medication. Information regarding the active ingredients and/or the prescribable synonym names may be retrieved. In examples, the prescribable synonym name may be the active ingredient name. As described herein, the drug information regarding the active ingredients and/or prescribable synonyms may include side effects, patient information, mornings, recalls, dosage information, the strength of the medication, the strength of an active ingredient, interaction with other medications, and the like.

At 518, the drug display name and/or the drug information may be presented to the user. For example, the user may be asked to confirm that the drug display name is accurate. In an example, the user may be presented with information indicating how to use the medication identity. In an example, the user may be presented with information indicating side effects that may be associated with the identified medication. In an example, the user may be presented with information indicating a potential interaction with another medication.

The user may be presented with a summary of the active ingredients that the user has consumed. For example, it may be determined that the user has consumed two medications, and it may be determined that five active ingredients are associated with those two medications. It may be determined how much of the five active ingredients the user consumed from the two medications and the user may be presented with a total amount consumed for each of the five active ingredients.

At 520, it may be determined that the user has confirmed that the identification of the medication is accurate. If the user may not have confirmed the identification of the medication, another attempt at identifying the medication may be performed at 502. If the user confirms the medication's identification, the user may be asked to capture an image of the medication at 522.

At 522, a user may capture an image of the medication, such that the image may assist in reminding the user of the medication. As described herein, a photo of a medication the user is using may allow the user to remember better which medication they record in a medication diary.

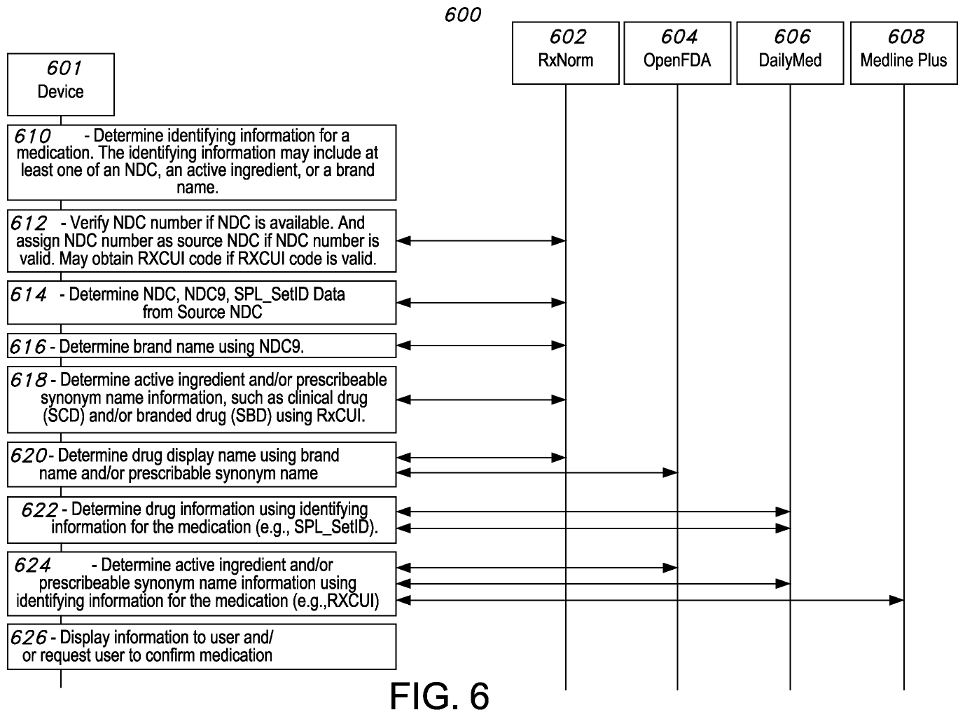

FIG. 6 depicts another example method for capturing medications and/or medication usage. As shown in FIG. 6, method 600 may include one or more of 610, 612, 614, 616, 618, 620, 622, 624, and 626 to capture a medication and/or medication usage. As shown in FIG. 6, method 600 may be performed by device 601, which may be any of the devices disclosed herein, such as device 204 shown with respect to FIG. 6. Device 601 may comprise components 120 shown with respect to FIG. 1.

Referring again to FIG. 6, at 610, device 601 may determine identifying information for a medication. The identifying information may include at least one of an NDC, a regulatory identifier, an active ingredient, a brand name, a generic name, a combination thereof, and/or the like. The medication may be an over-the-counter medication or a prescription medication. Device 601 may determine identifying information for the medication by performing a dynamic capture of the medication, such that information (e.g., text) may be retrieved from an image or video. Device 601 may determine identifying information by using artificial intelligence to identify the medication. Device 601 may determine an NDC associated with the medication.

At 612, device 601 may verify the NDC number if the NDC is available. For example, if an NDC number has been identified, device 601 may use RxNorm 602 to query a database to determine if the NDC is valid. If the NDC is determined to be valid, device 601 may assign the NDC as a source NDC.

Device 601 may use RxNorm 602 to retrieve information regarding the medication using the NDC. The information may include another medication identifier, a database identifier, medication information, information regarding side effects, the strength of the medication, and/or the like. The information may include an RxCUI, which may be a database identifier and/or a medication identifier.

At 614, a first NDC may be used to determine a second NDC. Device 601 may translate an NDC from one NDC format to another NDC format. This may be done, for example, to retrieve data from one or more databases that may use different NDC formats. For example, the NDC may be a source NDC in a 10 or 11-digit format. The source NDC may be converted to a 9-digit format, and the 9-digit format may be used to retrieve information from one or more databases.

To retrieve available information from one or more databases, device 601 may use a first NDC (e.g., NDC10 or NDC11) to determine a second NDC (NDC9), where the first NDC may be in a different format than the second NDC. For example, a source NDC may generate a second NDC that may be one of a 9-digit NDC format, a 10-digit NDC format, or an 11-digit NDC format. The NDC format may be a character format of any number, such as a 12-character format.

Device 601 may use the first NDC and/or the second NDC to retrieve medication information using RxNorm 602. For example, device 601 may use a 9-digit format NDC to determine a SPL ID (e.g., SPL_SetID). As another example, device 601 may use a SPL ID, which may be associated with an NDC, to retrieve medication information using RxNorm.

At 616, device 601 may determine a brand name. For example, device 601 may determine a brand name using an NDC in a 9 digital NDC format (e.g., NDC9). Device 601 may use RxNorm 602 to determine data regarding a brand name for an NDC (e.g., NDC9) associated with the medication. Device 601 may receive data using RxNorm 602 and analyze the data to determine the brand name associated with the NDC.

At 618, an active ingredient and/or prescribable synonym name, a clinical drug (SCD) and/or a branded drug (SBD), may be determined. Device 601 may use a medication identifier and/or a database identifier associated with the medication, such as a RxCUI, to determine one or more active ingredients that are used in the medication. For example, device 601 may use RxNorm 602 to query and/or retrieve an active ingredient, a prescribable synonym name, a clinical drug, and/or a branded drug. The one or more active ingredients may have a prescribable synonym name. Device 601 may determine the prescribable synonym name and may retrieve information related to the prescribable synonym name. Information regarding the active ingredient and/or the prescribable synonym name may include warnings, recalls, benefits provided by the drug, side effects that may be caused by the drug, diseases that may be treated by the drug, patient information, prescription information, treatment information, a combination thereof, and/or the like.

At 620, a drug display name may be determined using a brand name and/or a prescribable synonym name. A drug display name may indicate to a user which active ingredients may be included in the medication. A drug display name may indicate the brand name and one or more active ingredients. A drug display name may indicate the brand name, an active ingredient, and the amount for the active ingredient. A drug display name may indicate the brand name, a generic name, a prescribable synonym name, an active ingredient, an amount for the active ingredient, an amount for the brand name medication, a combination thereof, and/or the like. In an example, device 601 may interact with RxNorm 602 and/or OpenFDA 604 to query and/or retrieve the drug display name or data associated with the drug display name.

At 622, drug information may be determined using identifying information from the medication. Device 601 may have determined one or more medication identifiers, such as an NDC, a RxCUI, a SPL ID, and/or the like. Device 601 may send a query to OpenFDA 604 to retrieve information regarding the identified medication, such as a SPL ID. Device 601 may send a query to DailyMed 606 to retrieve information regarding the identified medication. For example, device 601 may retrieve prescribing information, boxed warning information, indications and usages for the medication, dosage and administration for the medication, contraindications for the medication, warnings and precautions for the medications, adverse reactions to the medication, drug interactions to the medication, overdosage information, a description of the medication, clinical pharmacology for the information, non-clinical toxicology, clinical studies, storage and handling information, SPL information, display panel information, ingredients and appearance information, manufacture information, marketing status, a Drug Enforcement Agency (DEA) schedule, packager information, a combination thereof, and/or the like.

At 624, active ingredient and/or prescribable synonym information may be determined using identifying information for the medication, such as a RxCUI. The active ingredient and/or prescribable synonym information may be received from a database associated with the National library of medicine (e.g., DailyMed 606, and Medline Plus 608). For example, device 601 may send a query to retrieve the information from OpenFDA 604, DailyMed 606, and/or Medline Plus 608. The information may include articles regarding the active ingredient and/or prescribable synonym name. An example of the information may be seen in FIG. 7.

Referring again to FIG. 6, at 626, information may be displayed to a user and/or user may be requested to confirm the identification of the medication. For example, device 601 may present the identification of the medication to the user and may request that the user confirm that the identification of the medication is correct. Device 601 may present information retrieved from one or more databases to the user. Device 601 may provide a summary of the medication and the amount of active ingredients included in the medication to the user. An example of the information may be seen in FIG. 7.

FIG. 7 depicts an example of information regarding a medication that may be displayed to a user. The information may be medication information 700. The medication information 702 may include medication details 702 and/or drug interactions 704. Medication details 702 may include medication details such as the drug name and strength at 710. For example, at 710, the user may be informed that the identity of the medication is Tylenol extra strength 500 mg oral tablets. The information may include an NDC code at 710. For example, the user may be informed that the NDC code may be in an 11-digit format in NDC and may be provided with the NDC. The information may include a picture of the medication from the manufacturer, FDA, regulatory authority, combination thereof, and/or the like. The picture may assist the user in confirming that the medication has been identified. Information may include dosing and reminders at 710. For example, if the user may be informed as to how the medication may be used. Information may include links and/or information from DailyMed at 706 and/or information from MedlinePlus at 708.

Information may include potential drug interactions, such as drug interactions 704, with other medications that a device may have determined that the user may be taking. The warnings regarding potential drug interactions may be received and were determined by the National Library of Medicine, the FDA, a regulatory authority, and/or the like.

FIG. 8 depicts an example method for determining an amount of an active ingredient included in a medication. As shown in FIG. 8, method 800 may include one or more of 808, 810, 814, and/or 816 to determine an amount of an active ingredient that may be included in the medication. As shown in FIG. 8, method 800 may be performed by device 801, which may be any of the devices disclosed herein, such as device 204 shown with respect to FIG. 6. Further, device 601 may comprise components 120 shown with respect to FIG. 1.

In an example, a device may be used to determine a summary of one or more active ingredients that a user, such as a patient, may have consumed. The summary may be referred to as a dosage summary, a medication summary, an ingredient summary, and/or a dosage summary by active ingredient. A user may use the device to take an image or video of a medication. A medication label may be determined from the image or video of the medication. The medication label may include information regarding the medication, such as a medication identifier.

Referring again to FIG. 8, at 808, a first medication identifier may be determined. The first medication identifier may be an NDC. The first medication identifier may be determined from an image and/or video of the medication that may include a medication label. For example, the medication label may indicate an NDC. The first medication identifier may be confirmed against a database and may be assigned as a source NDC and/or source medication identifier. For example, the first medication identifier may be identified and/or confirmed by sending a request at 809 using RxNorm 802. At 811, RxNorm 802 may respond by sending a response to device 801. In an example, the response may indicate that the identification is confirmed. In an example, the response may indicate that the identification was unable to be confirmed.

At 808, a request may be sent to one or more databases using an API, such as RxNorm 802. The request may indicate a request for information associated with the first medication identifier (e.g., the NDC) from one or more databases. The one or more databases may include a database, such as a database from the National Institute of Health and/or a database from the National Library of Medicine. In response to a request, device 801 may receive the requested information, which may include a second medication identifier, such as an NDC, a RxCUI, and/or the like. The requested information may include a mapping of the first medication identifier to the second medication identifier.

At 810, device 801 may determine a second medication identifier for a medication. Device 801 may determine the second medication identifier using the first medication identifier. Device 801 may determine the second medication identifier by analyzing data received at 808.

For example, at 809, device 801 may send a request to a database using an API, such as RxNorm. The request may include an NDC. At 811, the database may send a response to device 801. The response may include a second medication identifier, such as RxCUI. At 810, device 801 may determine that the second medication identifier is the RxCUI and may send a request to a database using an API, such as RxNorm 802 at 812. The database may send a response to the device 801 at 813. The response may include medication information associated with the RxNorm. The medication information may include a brand name, an indication of a clinical drug, an indication of a brand drug, one or more active ingredients associated with the medication and/or second medication identifier (e.g., RxCUI), a strength factor, an indication of a drug pack, an indication of a brand pack, a name associated with an active ingredient, a unit strength, a combination thereof, and/or the like.

At 817, device 801 may verify a second medication identifier, such as an RxCUI, by sending a request to one or more databases using an API, such as RxNorm 802. At 818, device 801 may receive a response from one or more databases. The response may include medication information and may include a confirmation that the RxCUI is associated with the medication. For example, the response may indicate that the RxCUI is valid and/or valid for a medication.

At 814, an active ingredient associated with the medication may be determined. A medication may be comprised of one or more active ingredients. Using the first medication identifier and/or the second medication identifier, device 801 may determine one or more medication ingredients. Device 801 may determine an amount of the active ingredient that may be included in the medication. Device 801 may do this for one or more (e.g., each active ingredient) associated with the medication such that device 801 may have a summary of how much of an active ingredient (e.g., each active ingredient) is included in the medication.

Device 801 may determine whether the medication may be a single-ingredient medication, a multiple-ingredient medication, and/or a medication provided in a drug pack. Device 801 may determine a strength associated with the medication and/or an active ingredient in the medication.

At 816, device 801 may display a summary to a user. The summary may include a list of the active ingredients in a medication. The summary may indicate the amounts of the active ingredient in the medication.

FIG. 9A depicts an example user interface that lists one or more identified medications. The data shown in FIG. 9A may be determined, calculated, and/or displayed by a device. The data may include a subject id at 902, a date at 904, an NDC at 906, a quantity at 908, a unit at 910, a display name at 912, one or more ingredients at 914, a dosage at 916, a dose unit at 918, a combination thereof, and/or the like. For example, a device may determine one or more active ingredients for a medication, the concentrations associated with the one or more active ingredients, the amounts of the one or more active ingredients, a combination thereof, and/or the like.

In an example, the user interface may display a summary. The summary may be referred to as a dosage summary, a medication summary, an ingredient summary, and/or a dosage summary by active ingredient.

As shown in FIG. 9A, a display name may be determined for a medication at 912. The display name for the medication may comprise a brand name for the medication, a generic name for the medication, an active ingredient associated with medication, a strength of the medication, a strength of an active ingredient associated with the medication, a combination thereof, and/or the like.

As shown in FIG. 9A, one or more active ingredients for a medication may be determined at 914. A dose and/or strength for the one or more active ingredients may be determined at 916. A unit for the one or more active ingredients may be determined at 918. For example, it may be determined that an extra-strength headache relief medication comprises 130 mg of caffeine, 500 mg of aspirin, and 500 mg of acetaminophen.

A date when the medication may have been consumed may be recorded at 904. An NDC for the identified medication may be recorded at 906. A quantity of the medication and a unit of the medication may be provided at 908. For example, it may be determined that extra strength headache relief medication may be a tablet, and two tablets may have been consumed.

FIG. 9B depicts an example user interface that summarizes one or more active ingredients associated with one or more medications. The data shown in FIG. 9B may be determined, calculated, and/or displayed by a device. The data may include a subject identification at 920, a date at 922, one or more ingredients at 924, a count at 926, a mean at 928, a sum at 930, a min at 932, a max at 934, a combination thereof, and/or the like. The summary may be referred to as a dosage summary, a medication summary, an ingredient summary, and/or a dosage summary by active ingredient.

In an example, a user may consume one or more medications throughout the day. A medication (e.g., of one or more medications) may be comprised of one or more active ingredients. To assist a user in understanding how much of an active ingredient they are consuming, the one or more medications may be broken down into one or more active ingredients at 924, and an amount of an active ingredient for each of the one or more medications may be determined. At 930, a sum of the active ingredients may be presented to a user so that they may understand how much of the active ingredient they have consumed within a timeframe. The timeframe may be a day, month, week, year, and/or the like.

As shown in FIG. 9B, the active ingredients may be listed according to a timeframe, such as a date at 922. Information regarding an active ingredient may be shown. A count of how many times the active ingredient may have been consumed in a day may be provided at 926. A mean of the amount of the active ingredient may be provided at 928. This may indicate to a user the mean amount of the active ingredient they consume per count. A sum of the amount of active ingredient may be provided to a user at 930. A minimum amount of the active ingredient from a medication consumed by the user may be provided to the user at 932. A maximum amount of the active ingredient from a medication consumed by the user may be provided to the user at 934.

Embodiments herein may provide for the aggregation and analysis of medication data. FIG. 9C depicts an example user interface that summarizes one or more active ingredients associated with one or more medications. For example, the data show in FIG. 9C may include a sample of medication diary data and may include a subject identification at 936, a date at 938, a NDC at 940, a RxCUI at 942, a quantity at 944, a unit at 946, a medication name at 948, one or more ingredients at 950, one or more doses at 952, one or more dose units at 954, a combination thereof, and/or the like.

In an example, NDCs may be mapped to RxCUIs, which provide detailed information on ingredient, dose (as known as strength) and dose unit. The data may be mapped into various drug classes such as Anatomical Therapeutic Chemical (ATC) classification to address analysis requests. Aggregation and analysis at the patient level or study level may be performed by drug, ingredient, or class, using RxNorm and linkage to other drug dictionaries. Medications entered as free text may be mapped using an algorithm, which may be incorporated as an endpoint of the RxNorm API.

FIG. 9D depicts an example user interface that provides a daily dosage summary for one or more active ingredients associated with one or more medications. The data shown in FIG. 9D may include a subject identification at 956, a date at 958, a NDC at 960, a RxCUI at 962, a quantity at 964, a unit at 966, a medication name at 968, one or more ingredients at 970, one or more doses at 972, one or more dose units at 974, a combination thereof, and/or the like.

As shown in FIG. 9D, embodiment herein may determine how much medication and/or how much of an activity ingredient a user may have consumed within a period. For example, a summary may be determined for an hourly dosage (e.g., 8 hours, 12 hours, 23, hours, 48 hours, etc.), a daily dosage, a weekly dosage, a monthly dosage, a combination thereof, and/or the like. The dosage may be for an active ingredient. For example, a daily dosage for acetaminophen may be determined.

How many active ingredients a user may have consumed may be determined. How many of those active ingredients the user may have consumed may be determined. For example, if the user consumes a cold medication with three active ingredients and a headache medication with two active ingredients, a summary of the five active ingredients may be provided.

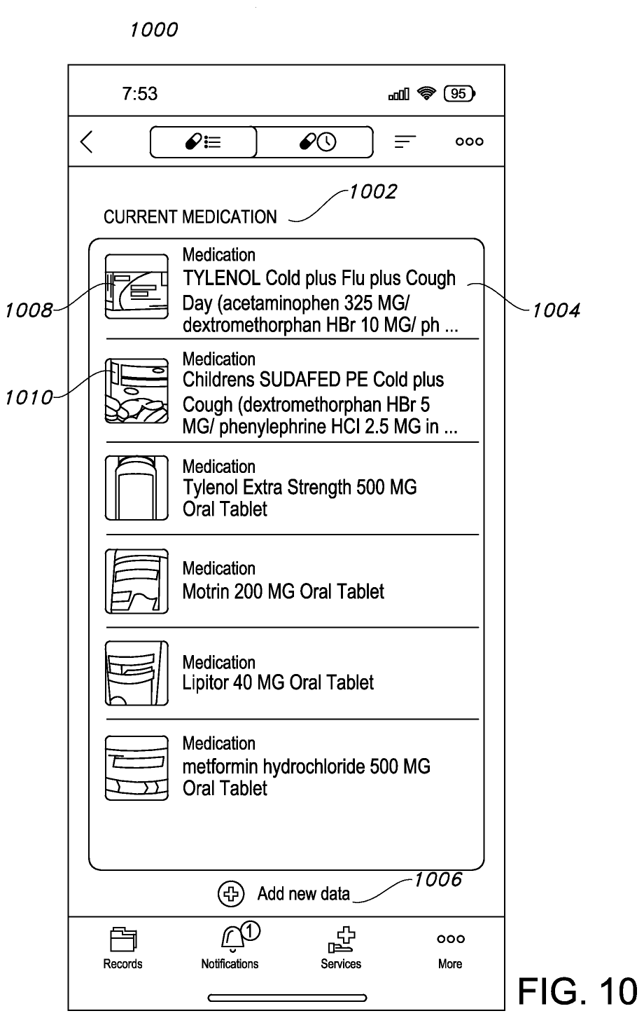

FIG. 10 depicts an example user interface that lists one or more medications associated with a patient. As shown in FIG. 10, interface 1000 may be an interface that may list medications that a patient may be consuming or may have consumed. For example, interface 1000 may display medication the user may have used in the past. As another example, at 1002, interface 1000 may display medications the user is using or may have been prescribed. At 1006, a user may add a medication to the list of medications.

In an example, at 1004, the list of the one or more medications may include photos of the one or more medications, such as at 1008 and 1010. For example, a user may take a photo of a medication and add the photo to the list of the one or more medications. The photo may assist the user in remembering the medication. The photo may be a photo of the package of the medication, a label of the medication, a photo of the medication, a photo of a bottle containing the medication, and/or the like. The photo of the medication may be more memorable to a user than the name of the medication. The photo may be of an item and/or object that may help the user identify the medication. For example, the photo may be a photo of a note that the user associates with the medication. As another example, the photo may be a photo of a location where the user stores the medication.

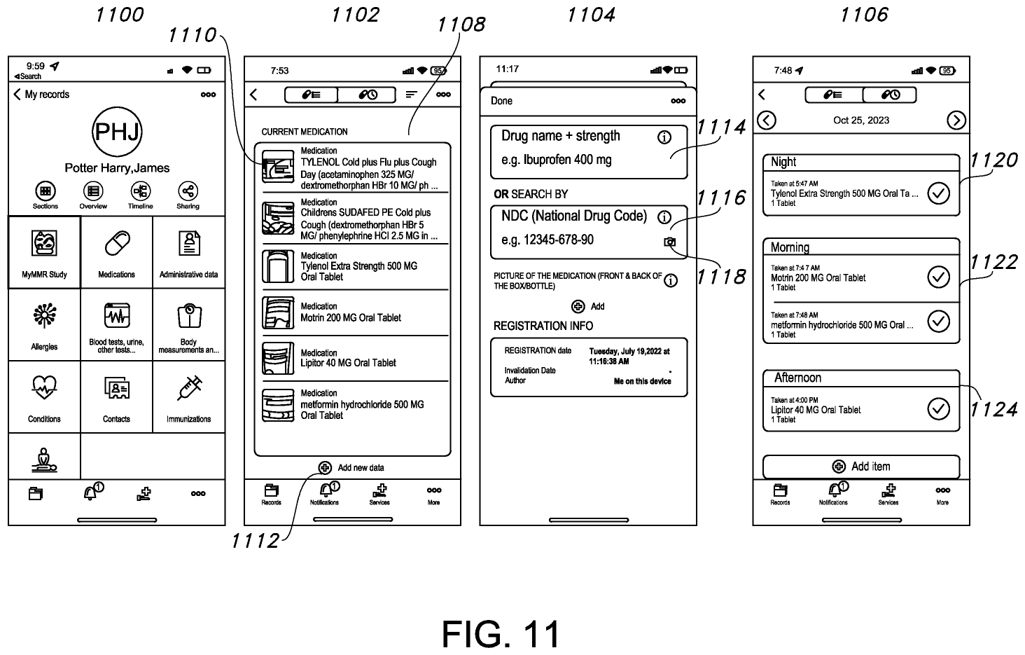

FIG. 11 depicts example user interfaces for capturing medications and/or medication usage. At 1100, a user may initiate the start of a program for capturing medication and/or medication usage. As shown at 1100, the user may have the ability to record their medications (e.g., a medication diary), identify a medication, provide and/or review patient information, provide and/or review information regarding medical conditions, provide and/or review information regarding allergies, a combination thereof, and/or the like.

At 1102, a user may be presented with one or more medications that have been identified. The identified medications, such as the list shown at 1108, may include a photo, such as at 1110, that the user may have captured. The photo may assist the user in remembering the medication. For example, a photo of the package of the medication or a label of the medication may be more memorable to a user than the name of the medication. As shown at 1112, the user may add a new medication.

At 1104, a user may be presented with an interface to add a medication. The user interface may allow the user to identify the medication using a medication identifier, such as an NDC, a brand name, and/or an active ingredient. In an example, a user may enter the medication identifier at 1114 and/or at 1116. For example, at 1118, a user may take a photo and/or video of a medication label and the medication may be identified using information from the photo and/or video as described herein.

At 1106, the user may be presented with an interface to indicate what medications the user may have consumed. The user interface may give the user the ability to know when they took the medication. For example, the user interface may indicate that a medication was consumed in the morning at 1122, night at 1120, or afternoon at 1124. The user interface may indicate a display name for the medication. The display name for the medication may comprise a brand name for the medication, a generic name for the medication, an active ingredient associated with medication, a strength of the medication, a strength of an active ingredient associated with the medication, a combination thereof, and/or the like.

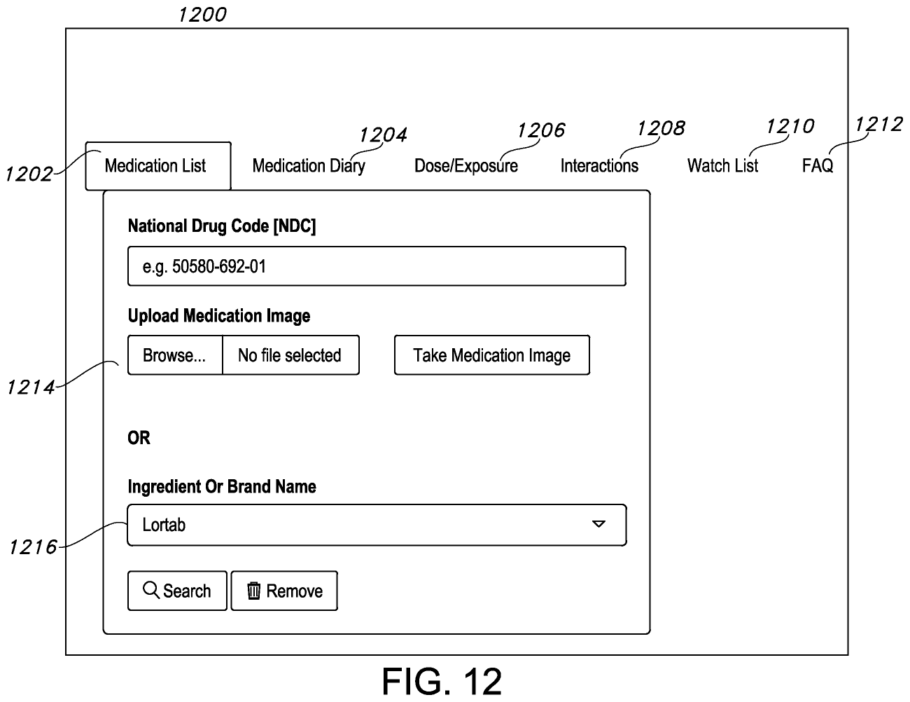

FIG. 12 depicts an example user interface for capturing medications and/or medication usage for a medication list. Interface 1200 may include medication list module at 1202, medication diary at 1204, dose/exposure module at 1206, interactions module at 1208, watch list module at 1210, and frequently asked questions module at 1212.

Interface 1200 may include a medication list module 1202. Medication list module 1202 may be an interface for medication list 317 described with respect to FIG. 3. Referring again to FIG. 12, medication list module 1202 may provide a repository for one or more medications that a user is taking or has taken in the past. It may provide a view of the user's medication history, which may be helpful for healthcare providers in understanding the user's health condition and planning future treatments.

At 1214, a user may take a photo or upload an existing photo to identify a medication for the medication list module 1202. This may provide a visually intuitive method for users to add medications to their list and may be helpful when dealing with multiple medications with complex names.

At 1216, a user may have the capability to search for a medication by its name, active ingredient, or brand name. This search function may assist the user in locating medication information and may allow for quick and accurate updates to their medication list in module 1202. The search function may help users to learn more about their medications, including potential interactions, side effects, and dosages.

Interface 1200 may include medication diary module 1204. Medication diary module 1204 may be an interface for medication diary 320, described with respect to FIG. 3. Referring again to FIG. 12, medication diary module 1204, may allow a user to record their medication usage over a duration of time. For example, a user may record when they take a medication, the dosage, and any side effects they experience. This may provide information about the effectiveness of medications and their impact on the user's health.

Interface 1200 may include dose/exposure module 1206. Dose/exposure module 1206 may be an interface for dosage summary 322, described with respect to FIG. 3. Referring again to FIG. 12, dose/exposure module 1206, may allow a user to monitor the amount of medication that the user is exposed to. It may calculate dosages based on the information provided by the user or the healthcare provider. This may help to ensure that the user is not over- or under-exposed to any medication, which may help prevent adverse drug reactions or ineffective treatment.

Interface 1200 may include interactions module 1208. Interactions module 1208 may be an interface for interactions 322, described with respect to FIG. 3. Referring again to FIG. 12, interactions module 1208, may allow a user to analyze the potential interactions between the medications that the user is taking. It may flag any possible negative interactions or synergistic effects. This may assist in preventing harmful drug interactions and may optimize the benefits of the medication regimen.

Interface 1200 may include watch list module 1210. Watch list module 1210 may be an interface for watch list 323, described with respect to FIG. 3. Referring again to FIG. 12, watch list module 1210 may provide a user with and/or may monitor a list of medications that the user should avoid. These may be medications that have been identified by a healthcare provider as potentially harmful for the user due to their interactions with other drugs the user may be taking. This module may help protect the user from adverse drug interactions.

Interface 1200 may include a frequently asked questions (FAQ) module 1212. FAQ module 1212 may retrieve information from medication information 318, shown with respect to FIG. 3. Referring again to FIG. 12, FAQ module 1212 may provide users with answers to questions about their medications. This may include information about side effects, dosages, timing, and potential interactions. FAQ module 121 may provide a reference for users, helping them to understand and manage their medications more effectively.

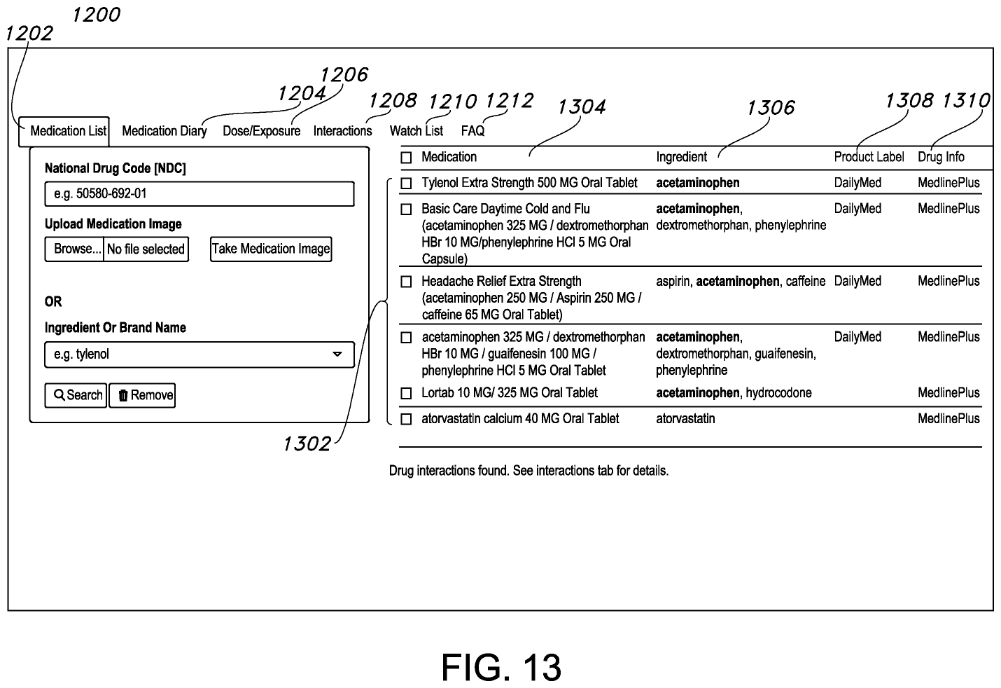

FIG. 13 depicts an example user interface for capturing medications and/or medication usage for a drug interaction. As described herein, interface 1200 may include watch list module 1210. In an example, data may be provided at 1302, which may comprise a variety of important information about one or more medications. This may include a medication name at 1304, which may allow a user to identify a medication. One or more ingredients may be listed at 1306, which may inform users about the active and inactive substances in a medication. A link to a product label may be provided at 1308. This link may offer a user access to information about the medication, including its manufacturer, directions for use, warnings, and/or the like. A link to drug information may be provided at 1310. This link may direct users to further detailed information about the medication, including its pharmacological class, common uses, potential side effects, drug interactions, and/or the like.

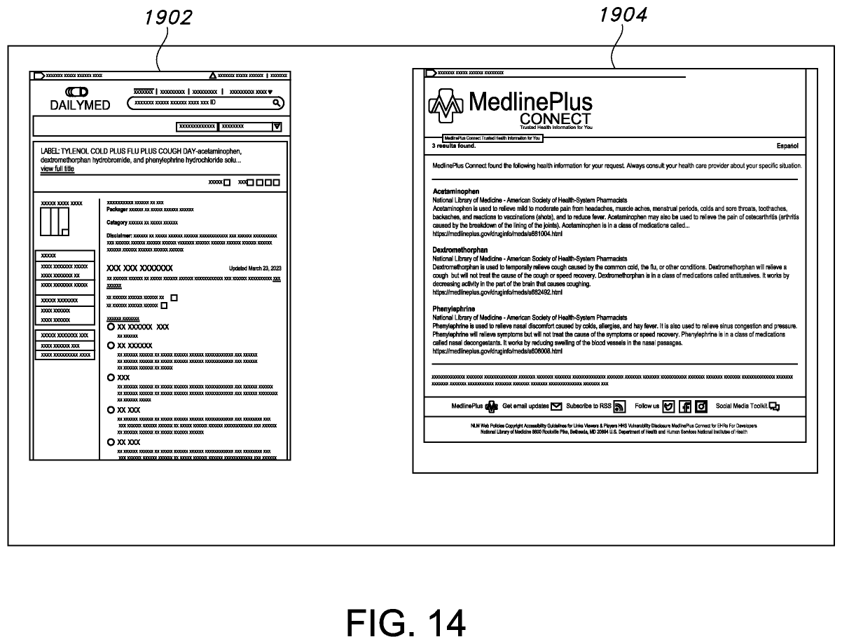

FIG. 14 depicts an example medication information. At 1402, product label information may be provided to a user. The product label information may be the medication information described herein. The product label information may be from a database such as DailyMed. The product label information may be the product label information provided for by the link at 1308, shown with respect to FIG. 13.

Referring again to FIG. 14, at 1404, drug information may be provided. The product label information may be the medication information described herein. The drug information may be from a database such as MedlinePlus. The drug information may be the drug information provided for by the link at 1310, shown with respect to FIG. 13.

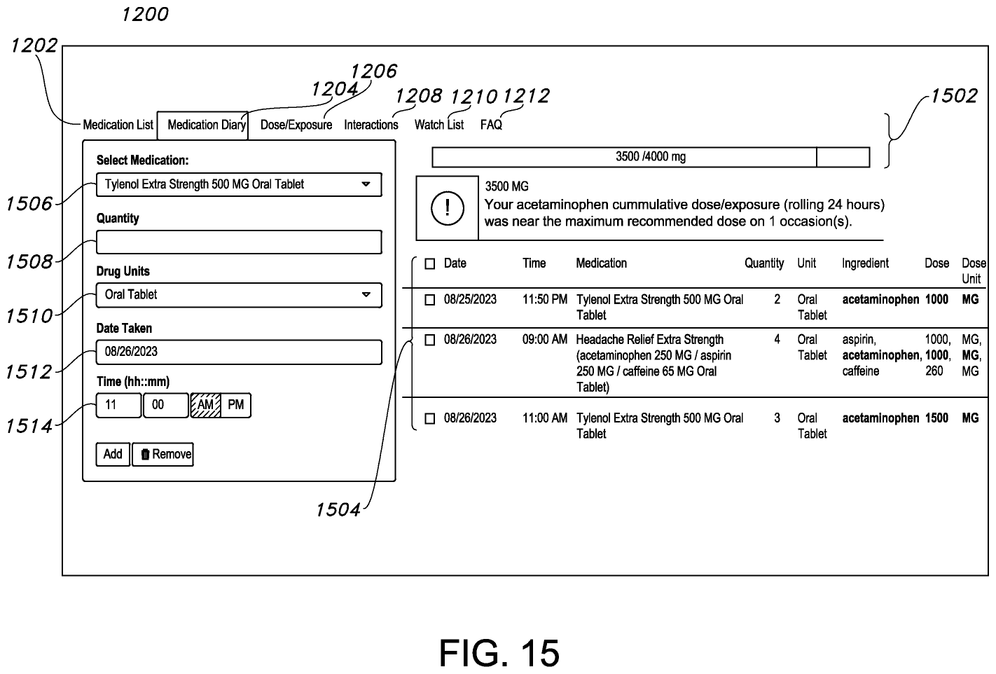

FIG. 15 depicts an example user interface for capturing medications and/or medication usage for a medication diary. For example, FIG. 15 illustrates an example user interface for documenting medications and their usage in a medication diary module 1204. User-provided data may be incorporated at 1504, which may include details such as the date and time of medication, the name of the medication, the quantity taken, the unit of measurement, one or more active ingredients, the dosage, and the unit of the dose.

For example, a user may enter the medication name at 1506, input the quantity at 1508, select the relevant unit at 1510, and record the date and time of consumption at 1512 and 1513, respectively. This approach may possibly assist in maintaining a record of medication usage, which may be beneficial for both the user and their healthcare providers.

A dose warning may be provided at 1502, which may signal that the user is nearing a threshold for a particular medication. For example, the user may be near a maximum recommended dosage of acetaminophen. The ability to flag such instances may aid in preventing overdoses or adverse reactions due to excessive medication intake.

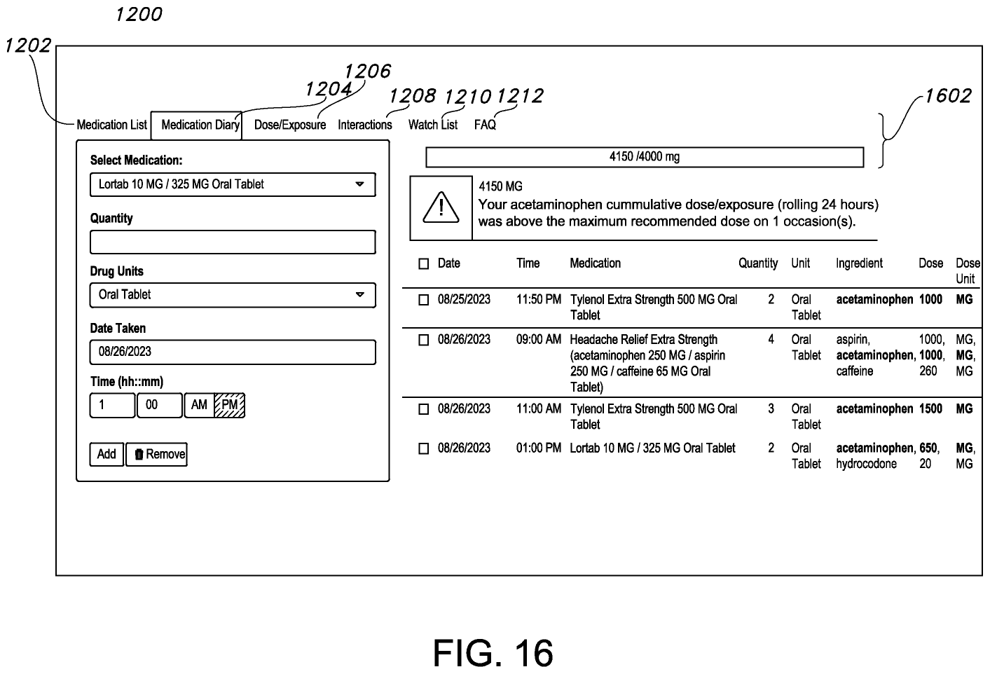

FIG. 16 depicts an example user interface for capturing medications and/or medication usage for a medication diary. At 1602, interface 1200 may alert the user that they have exceeded a threshold for a specific medication. For instance, the user may have surpassed the maximum advisable quantity of acetaminophen for a given time period.

In an example, the interface may generate warnings and instructions to the user. The warnings may include messages such as “Warning: You have exceeded the recommended daily limit of acetaminophen. Excessive intake may lead to liver damage.” The system may provide instructions like “Please discontinue use and consult with your healthcare provider.”

In addition to these user-facing alerts, the system may notify a healthcare professional associated with the user. This may be done automatically or upon the user's confirmation. The healthcare provider may take appropriate actions, such as contacting the patient for a consultation, adjusting the medication regimen, and/or arranging for immediate medical intervention.

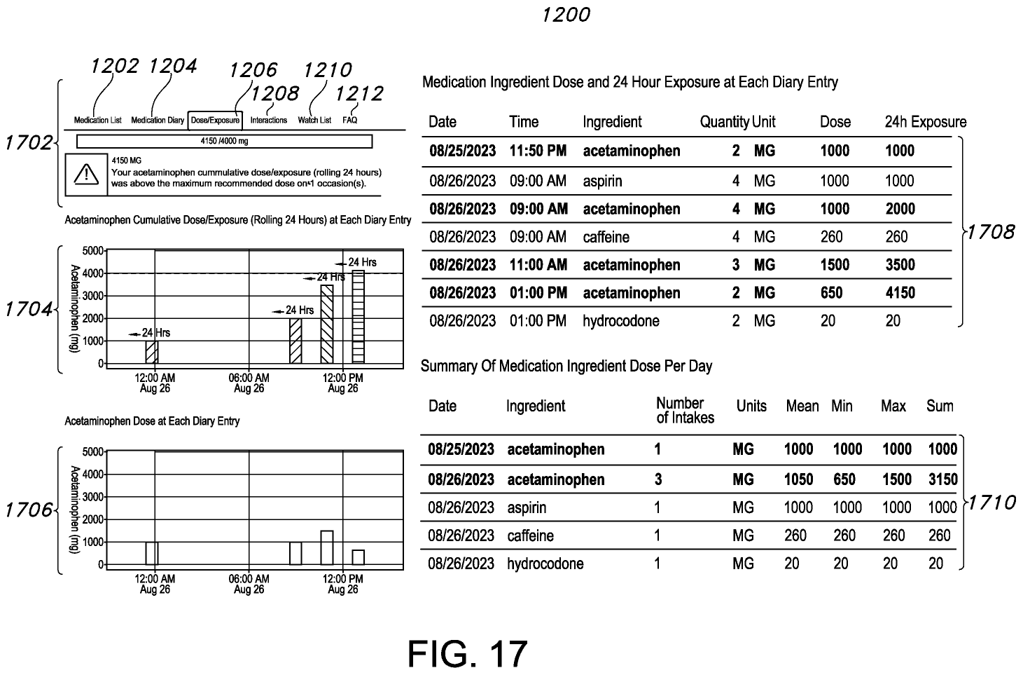

FIG. 17 depicts an example user interface for capturing medications and/or medication usage for a dose assessment or an exposure assessment. At 1702, interface 1200 user may provide a user with a notification indicating that the user may have exceeded a threshold for a medication. For example, the user may have surpassed the maximum recommended amount of acetaminophen for a given time period. This warning may be accompanied by instructions suggesting immediate actions, such as ceasing further intake of that medication, drinking plenty of fluids to help flush out the excessive drug, seeking prompt medical attention, and/or the like.

Interface 1200 may provide recommendations for future actions, such as advice on how to avoid similar incidents in the future. For example, the user may be advised to double-check dosages before taking medications, to avoid taking multiple products containing the active ingredient, to consult with a healthcare provider before starting new medications, a combination thereof, and/or the like.

At 1704, interface 1200 may present a graph that plots cumulative dose or exposure over a time period, such as a 24-hour cycle. This visual representation may allow a user to understand their medication usage patterns and identify any periods of excessive intake.

At 1706, interface 1200 may provide graph that may illustrate the quantity of medication taken (e.g., displayed on the y-axis) against a time period (e.g., shown on the x-axis), such as the dates of diary entries. This may aid a user in visualizing their medication intake over time, which may be beneficial in managing their medication schedules and adhering to prescribed dosages.

At 1708, a user may be provided with data detailing the exposure to the medication over a time period. This data may include details such as the date, time, ingredients, quantity, unit, dosage, total exposure over a time period, a combination thereof, and/or the like.

1710, interface 1200 may present a summary of medication ingredients dosage per day. This data may include the date, one or more ingredients, a number of intakes, units, a mean, a minimum, a maximum, a sum, a combination thereof, and/or the like.

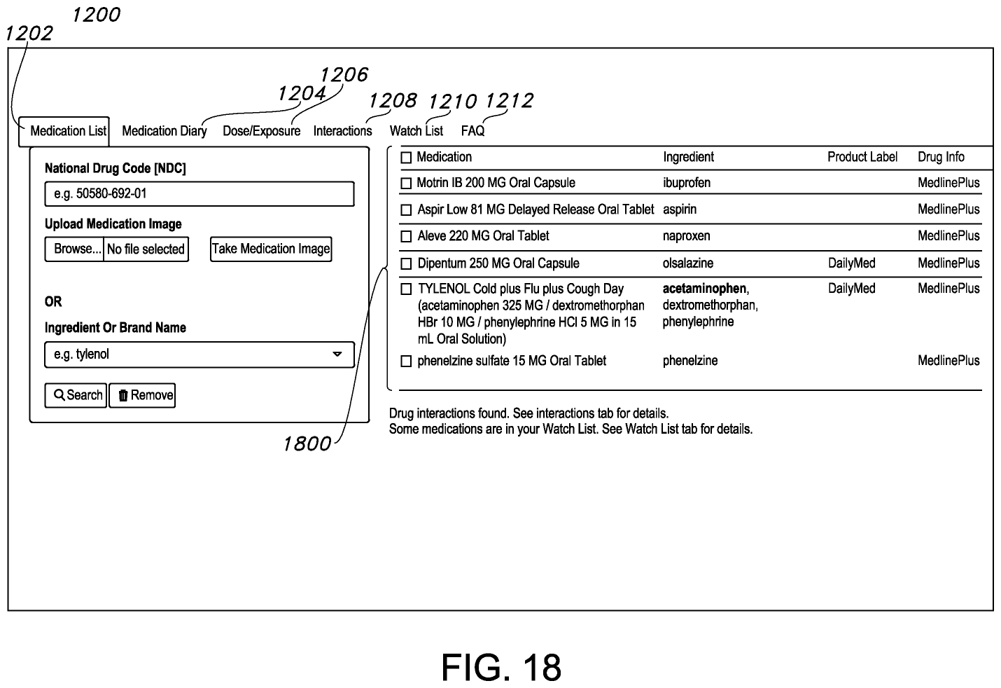

FIG. 18 depicts an example user interface for capturing medications and/or medication usage for a medication watch list. In an example, interface 1200 may display data associated with watch list module 1210, which may include displaying data at 1800. The data at 1800 may include the name of the medication, one or more active ingredients, and medication information. The medication information may further include a product label, a link to a product label, drug information, a link to drug information, a combination thereof, and/or the like. The data at 1800 may provide a user with a view of their medication watch list, providing an information regarding one or more medications that a healthcare provider wants the user to monitor.

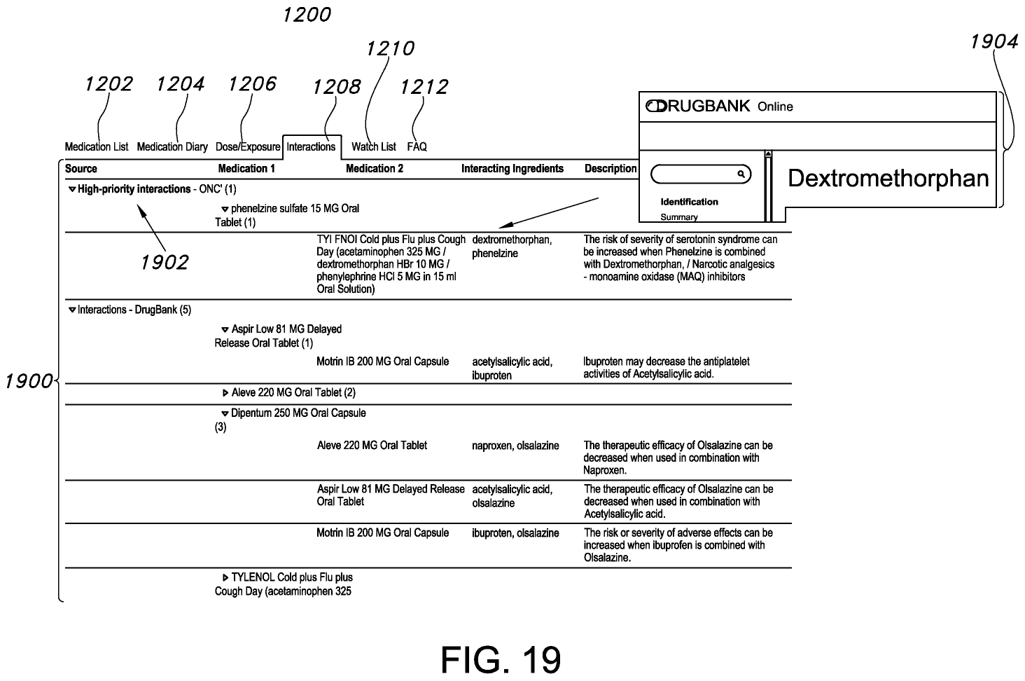

FIG. 19 depicts an example user interface for capturing medications and/or medication usage for a drug interaction. The interaction module 1208, as part of Interface 1200, may analyze the list of medications a user is taking and identify potential interactions between them. This may include both positive and negative interactions and may involve assessing the effects of different combinations of medications on the user's health.

Interface 1200 may display data at 1900, which may include a source of information, the names of a first and second medication, one or more interacting ingredients, a description of the interaction, a combination thereof, and/or the like.

This data may be organized based on priority. For example, at 1902, interactions deemed high priority may be listed before those categorized as lower priority. This prioritization may help users and healthcare providers to quickly identify and address interactions.

The data presented at 1900 may indicate an interaction between the first and second medications. The interacting ingredients may be responsible for this interaction, and may be identified at 1904. Information about these interactions may be provided at 1904, which may include the nature of the interaction, the potential effects on the patient, recommended actions for the user or their healthcare provider, a combination thereof, and/or the like.

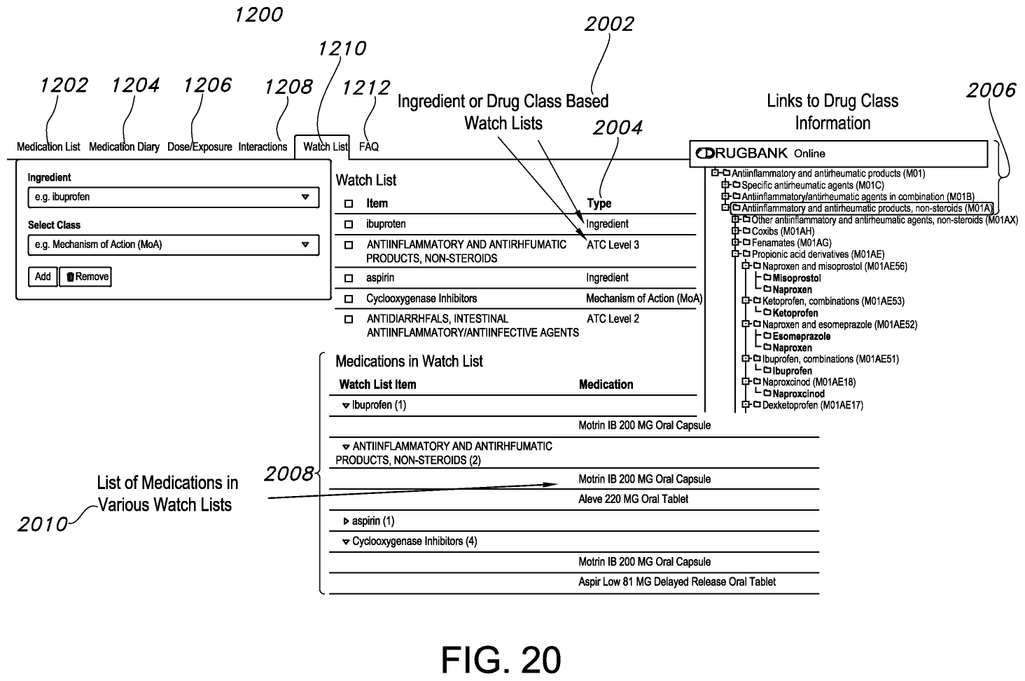

FIG. 20 depicts an example user interface for capturing medications and/or medication usage for a medication watch list. Interface 1200 may provide data generated by the watch list module 1210. At 2002, this data may include ingredients and/or a drug class on a watch list.

A drug class refers to a group of medications that work in a similar way or have a similar chemical structure. Grouping medications into classes may allow for better monitoring of medications that may have interactions. A drug class may be provided using a classification system, such as the Anatomical Therapeutic Chemical (ATC) classification system. The ATC classification system may be used to classify active ingredients of drugs based on the organ or system on which they act, their therapeutic and pharmacological properties, and/or their chemical characteristics. The ATC classification system may be used as a tool for drug utilization research and may aid in the monitoring and analysis of medication use and trends. In an example, a classification system, such as the ATC classification system, may organize medications (e.g., drugs) into different hierarchical levels or classes, starting with the anatomical group and then progressing through the therapeutic, pharmacological, and chemical subdivisions. This hierarchical structure may allow efficient categorization, and may facilitate a comparison of drug usage, research, and/or monitoring.

At 2004, a drug class or type may be listed, which may provide users with an understanding of the category their medication falls into. At 2006, information, such as medication information and/or links to medication information, may be provided for one or more drug classes. This may assist users in comprehending the nature of the medications they are advised to avoid.

At 2008, a list of medications may be provided by a healthcare provider. These may be medications that the user has been instructed to steer clear of due to potential adverse reactions or interactions with other medications they are taking.

At 2010, a list of other medications to avoid might be provided. This may be based on the user's history of potential interactions or other individual health considerations. For example, if a user has previously experienced adverse reactions to a certain class of drugs, medications from that class may be included in this list.

The foregoing detailed description has set forth various forms of the devices and/or processes via the use of block diagrams, flowcharts, examples, and/or embodiments. Insofar as such block diagrams, flowcharts, examples, and/or embodiments contain one or more functions and/or operations, it will be understood by those within the art that each function and/or operation within such block diagrams, flowcharts, and/or examples may be implemented, individually and/or collectively, by a wide range of hardware, software, firmware, or a combination thereof. Those skilled in the art will recognize that some aspects of the forms disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as a combination thereof. Those skilled in the art will appreciate that the mechanisms of the subject matter described herein are capable of being distributed as one or more program products in a variety of forms and that an illustrative form of the subject matter described herein applies regardless of the particular type of signal bearing medium used to carry out the distribution.

Instructions used to program logic to perform various disclosed aspects may be stored within a memory in the system, such as dynamic random-access memory (DRAM), cache, flash memory, or other storage. The instructions may be distributed via a network or by way of other computer-readable media. For example, instructions may be sent via a message and/or a signal. A computer-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, computer-readable storage used in the transmission of information over a network (e.g., the Internet) via electrical, optical, acoustical or other forms of propagated signals (e.g., carrier waves, infrared signals, digital signals, etc.). A non-transitory computer-readable medium may include any type of tangible computer-readable medium suitable for storing or transmitting electronic instructions or information in a form readable by a device (e.g., a computer).

As used herein, the terms “component,” “system,” “sub-system,” “module,” and the like may refer to a computer-related entity, a device, hardware, software, software in execution, a combination thereof, and/or the like. This application may refer to “determining” various pieces of information. Determining the information can include one or more of, for example, estimating the information, calculating the information, predicting the information, or retrieving the information from memory.

Additionally, this application may refer to “receiving” various pieces of information. Receiving is, as with “accessing,” intended to be a broad term. Receiving the information can include one or more of, for example, accessing the information, or retrieving the information (for example, from memory). Further, “receiving” is typically involved, in one way or another, during operations such as, for example, storing the information, processing the information, transmitting the information, moving the information, copying the information, erasing the information, calculating the information, determining the information, predicting the information, or estimating the information.

It is to be appreciated that the use of any of the following “/,” “and/or,” and “at least one of,” for example, in the cases of “A/B,” “A and/or B” and “at least one of A and B,” is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

We describe a number of examples. Features of these examples can be provided alone or in any combination, across various claim categories and types. Further, embodiments can include one or more of the following features, devices, or aspects, alone or in any combination, across various claim categories and types.

1. A device for determining a medication used by a patient, the device comprising:
a processor, the processor configured to:
determine label information for a medication;
determine a source national drug code (NDC) associated with the medication using the label information;
send a first message to a first server, wherein the first message indicates a request for a validation of the source NDC;
receive a second message from the first server, wherein the second message indicates the source NDC is valid, indicates a product NDC, and indicates a normalized identifier;
determine a brand name associated with the medication using the product NDC;
determine one or more active ingredients associated with the brand name using the normalized identifier;
determine a drug display name using the brand name and the one or more active ingredients;
determine active ingredient information for the one or more active ingredients; and
display the drug display name and the active ingredient information to a user.

2. The device of claim 1, wherein the normalized identifier is a RxNorm concept unique identifier (RxCUI).

3. The device of claim 1, wherein the product NDC comprises a labeler code and a product code.

4. The device of claim 1, wherein the source NDC is a package NDC, and wherein the package NDC comprises a labeler code, a product code, and a package code.

5. The device of claim 1, wherein the source NDC comprises a labeler code, a product code, and a package code, and wherein the product NDC comprises the labeler code and the product code.

6. The device of claim 1, wherein the processor is configured to determine the label information for the medication by receiving data from a user, wherein the data includes at least one of the source NDC, an active ingredient, a brand name, an image, or a video.

7. The device of claim 1, wherein the processor is configured to determine the brand name using the product NDC by:
sending a brand name request message to a second server, wherein the brand name request message indicates a request for a brand name associated with the product NDC; and
receiving a brand name response message from the second server, wherein the brand name response message indicates the brand name.

8. The device of claim 1, wherein the processor is configured to determine the one or more active ingredients associated with the brand name by:
sending an ingredient request message to the first server, wherein the ingredient request message indicates a request for one or more active ingredients associated with the normalized identifier;
receive an ingredient response message from the first server, wherein the ingredient response message indicates the normalized identifier and indicates one or more active ingredients; and
store the one or more active ingredients within a database such that the one or more active ingredients are associated with the brand name.

9. A method performed by a device for determining a medication used by a patient, the method comprising:
determining label information for a medication;
determining a source national drug code (NDC) associated with the medication using the label information;
sending a first message to a first server, wherein the first message indicates a request for a validation of the source NDC;
receiving a second message from the first server, wherein the second message indicates the source NDC is valid, indicates a product NDC, and indicates a normalized identifier;
determining a brand name associated with the medication using the product NDC;
determining one or more active ingredients associated with the brand name using the normalized identifier;
determining a drug display name using the brand name and the one or more active ingredients;
determining active ingredient information for the one or more active ingredients; and
displaying the drug display name and the active ingredient information to a user.

10. The method of claim 9, wherein the normalized identifier is a RxNorm concept unique identifier (RxCUI).

11. The method of claim 9, wherein the product NDC comprises a labeler code and a product code.

12. The method of claim 9, wherein the source NDC is a package NDC, and wherein the package NDC comprises a labeler code, a product code, and a package code.

13. The method of claim 9, wherein the source NDC comprises a labeler code, a product code, and a package code, and wherein the product NDC comprises the labeler code and the product code.

14. The method of claim 9, wherein determining the label information for the medication comprises receiving data from a user, wherein the data includes at least one of the source NDC, an active ingredient, a brand name, an image, or a video.

15. A non-transitory computer-readable medium storing processor-executable instructions comprising:
determining an image or a video of a medication label; and
determining a source national drug code (NDC) from the image, wherein the source NDC is associated with the medication label;
sending a first message to a first server, wherein the first message indicates a request for a validation of the source NDC;
receiving a second message from the first server, wherein the second message indicates the source NDC is valid, indicates a product NDC, and indicates a normalized identifier;
determining a brand name associated with the medication using the product NDC;
determining one or more active ingredients associated with the brand name using the normalized identifier;
determining a drug display name using the brand name and the one or more active ingredients;
determining active ingredient information for the one or more active ingredients; and
displaying the drug display name and the active ingredient information to a user.

16. The non-transitory computer-readable medium of claim 15, wherein processor-executable instructions for determining the drug display name using the brand name and the one or more active ingredients comprises:
determining a prescribable synonym name for at least an active ingredient of the one or more active ingredients; and
generating the drug display name by combining the brand name with the prescribable synonym name.

17. The non-transitory computer-readable medium of claim 15, wherein the processor-executable instructions for determining the active ingredient information for the one or more active ingredients comprises:
sending an active ingredient information request to a third server, wherein the active ingredient information request indicates at least one of a structured product labeling identification (SPL ID) or the normalized identifier; and
receiving an active ingredient information response, wherein the active ingredient information response indicates an active ingredient and indicates at least one of a warning, a direction regarding the active ingredient, or a usage direction, or a drug interaction warning.

18. The non-transitory computer-readable medium of claim 15, wherein the normalized identifier is a RxNorm concept unique identifier (RxCUI).

19. The non-transitory computer-readable medium of claim 15, wherein the product NDC comprises a labeler code and a product code.

20. The non-transitory computer-readable medium of claim 15, wherein the source NDC is a package NDC, and wherein the package NDC comprises a labeler code, a product code, and a package code.
